(19) United States
(12) Patent Application Publication
(10) Pub. No.: US 2021/0393854 A1
(43) Pub. Date: Dec. 23, 2021

(54) SKELETAL MUSCLE REGENERATION IN VOLUMETRIC MUSCLE LOSS USING BIOMIMETIC GLYCOSAMINOGLYCAN-BASED HYDROGEL

(71) Applicant: Purdue Research Foundation, West Lafayette, IN (US)

(72) Inventors: MENG DENG, West Lafayette, IN (US); Shihuan Kuang, West Lafayette, IN (US); Naagarajan Narayanan, Boulder, CO (US); Liangju Kuang, Quincy, MA (US)

(73) Assignee: Purdue Research Foundation, West Lafayette, IN (US)

(21) Appl. No.: 17/233,594

(22) Filed: Apr. 19, 2021

Related U.S. Application Data
(60) Provisional application No. 63/040,590, filed on Jun. 18, 2020.

(51) Int. Cl. A61L 27/52; A61L 27/26; A61L 27/36; A61L 27/38

(52) U.S. Cl. CPC A61L 27/52; A61L 27/26; A61L 27/3616; A61L 27/3633; C08L 5/00; A61L 27/3691; A61L 27/3817; A61L 27/3847; A61L 27/3891; A61L 27/3687

(57) ABSTRACT

Volumetric muscle loss (VML) injuries characterized by critical loss of skeletal muscle tissues result in severe functional impairment. Current treatments involving use of muscle grafts are limited by tissue availability and donor site morbidity. The present application relates to methods and composition matters for skeletal muscle healing and regeneration for a patient with volumetric muscle loss using a glycosaminoglycan-based hydrogel, wherein said hydrogel for skeletal muscle regeneration comprises functionalized hyaluronic acid (HA), functionalized chondroitin sulfate (CS) and poly(ethylene glycol) diacrylate (PEGDA), wherein said HA and said CS are cross-linked by said PEGDA.

### CROSS REFERENCE TO RELATED APPLICATIONS

The present U.S. patent application is related to and claims the priority benefit of U.S. Provisional Patent Application Ser. No. 63/040,590, filed Jun. 18, 2020, the contents of which are hereby incorporated by reference in their entirety into the present disclosure.

### GOVERNMENT SUPPORT CLAUSE

This invention was made with government support under AR068108 and AR071649 awarded by the National Institutes of Health. The government has certain rights in the invention.

### TECHNICAL FIELD

The present application relates to methods and composition matters for skeletal muscle healing and regeneration for a patient with volumetric muscle loss using a glycosaminoglycan-based hydrogel, wherein said hydrogel for skeletal muscle regeneration comprises functionalized hyaluronic acid (HA), functionalized chondroitin sulfate (CS) and poly(ethylene glycol) diacrylate (PEGDA), wherein said HA and said CS are cross-linked by said PEGDA.

### BACKGROUND

This section introduces aspects that may help facilitate a better understanding of the disclosure. Accordingly, these statements are to be read in this light and are not to be understood as admissions about what is or is not prior art.

Skeletal muscles have remarkable intrinsic ability to regenerate from minor injuries primarily due to satellite cells that reside on the surface of myofibers just beneath the basal lamina [1, 2]. However, the natural regeneration process is hindered in volumetric muscle loss (VML) injuries which are characterized by critical loss of skeletal muscle tissues resulting in severe functional impairment [3]. VML injuries are frequently observed in the lower limb with quadriceps VML being the most notable [4]. Current clinical treatments involving use of muscle grafts are limited by tissue availability and donor site morbidity. Thus, there is a pressing need to develop alternative strategies to promote functional regeneration of skeletal muscle tissues following severe injuries. There are unmet needs in the field of damaged muscle repair and regeneration.

### BRIEF DESCRIPTION OF THE DRAWINGS

The above and other objects, features, and advantages of the present invention will become more apparent when taken in conjunction with the following description and drawings wherein identical reference numerals have been used, where possible, to designate identical features that are common to the figures, and wherein:

FIG. 1A: Schematics illustrating synthesis and formation of HA-CS hydrogel. The carboxyl groups present in HA or CS were activated by EDC and NHS and then reacted with cystamine dihydrochloride. In the next step, the disulfide bonds were reduced by DTT reaction resulting in free thiol groups in the backbone of HA or CS. To form the hydrogel, HA and CS with thiol group were cross-linked with PEGDA at 37° C. via thiol-ene click chemistry. FIG. 1B: Representative light micrograph depicting the formed hydrogel. FIG. 1C: Rheological properties of HA-CS hydrogel with storage modulus (G′) as a function of time. The hydrogels showed a storage modulus G′ of 13.02±1.79 kPa from time sweep analysis (n=4). FIG. 1D: Representative cryo-SEM image of HA-CS hydrogel illustrating hydrogel pore structure, wherein a, b, and c denote the number of the respective repeating unit within polymer structure.

FIG. 2A: Schematics illustrating 3D cell encapsulation in the HA-CS hydrogel. In brief, C2C12 cells cultured in petri dishes were trypsinized, centrifuged, and pelleted. Individual components of the hydrogel were added to the pellet and mixed thoroughly. The mixture was added to syringe molds and incubated at 37° C. to induce gelation resulting in cell encapsulation within hydrogels. Representative confocal images of C2C12 cells via live/dead staining showing viability of encapsulated cells at FIG. 2B: day 1; FIG. 2C: day 3, and FIG. 2D: day 7.

FIG. 2E: MTS assay of encapsulated C2C12 cells indicating progressive cell growth during cell culture as evidenced from a significant increase in metabolic activity. Immunofluorescence staining of MF-20 for C2C12 cells with FIG. 2F: 0-day incubation and FIG. 2G: 3-day incubation in the GM illustrating improved myotube formation via 3-day incubation in the GM. FIG. 2H: qPCR illustrating a significant increase in MyoD, MyoG, and MYH8 of the encapsulated myoblasts for 3-day incubation in the GM compared to 0-day incubation. This further led to a significant increase in MYH8 after 4 days of differentiation. (*) denotes significant difference (p<0.05).

FIGS. 3A-3G: Schematics of the overall VML surgery procedure with implantation of HA-CS hydrogels. FIG. 3A: Animals were first shaved followed by skin incision to expose the quadriceps. FIG. 3B: A 4-mm biopsy punch was employed to create FIG. 3C: a full-length defect site. FIG. 3D: The treatment groups, autograft and HA-CS hydrogel (d) were implanted at the defect site. FIG. 3E: Suture was initiated at the muscle flap surrounding the defect site. FIG. 3F: The muscle flap was sutured to hold the implants at the defect site. FIG. 3G: the skin incision was sealed using tissue glue.

FIGS. 4A-4N: Gross morphology and histology comparison of sagittal sections obtained from the surgically injured quadriceps at 1-week (left three columns) and 4-week (right three columns) post-surgery in NT group (top row), AT group (middle row) and HA-CS group (bottom row). As observed by the macroscopic images, there were noticeable differences in the gross morphology of the injured quadriceps among the three treatment groups. In specific, the injury area is clearly visible from the gross morphology in the NT group and to a lesser extent in the AT group at 1-week and 4-week post-surgery. Notably, HA-CS group is virtually identical to the uninjured quadriceps at 4-week post-surgery. Representative H&E staining of the quadriceps sagittal section indicate very few newly regenerated fibers in the (FIGS. 4A-4B) NT group whereas greater numbers of newly regenerated central nuclei muscle fibers were found in (FIGS. 4C, 4D) AT and (FIGS. 4E, 4F) HA-CS groups at the injury site at 1-week post-surgery. At 4-week post-surgery, the defect site was distinctly observed in the (FIGS. 4G, 4H) NT group and to a lesser extent in (FIGS. 4I, 4J) AT group. Newly regenerated muscle fibers were observed near the boundary of the defect site in the AT group. Remarkably, the defect site was hardly observed in the (FIGS. 4K, 4L) HA-CS group with newly regenerated muscle fibers observed throughout the defect site. Black arrows indicate newly regenerating muscle fibers with central nuclei. (Scale bar: 2 mm for FIGS. 4A, 4C, 4E, 4G, 4I, 4K and 200 μm for FIGS. 4B, 4D, 4F, 4H, 4J, 4L). Histomorphometry analysis on the quadriceps sagittal sections indicating that the number of newly regenerated muscle fibers with central nuclei were significantly higher in AT and HA-CS groups compared to NT group at (FIG. 4M) 1-week and (FIG. 4N) 4-week post-surgery. (*p<0.05 as compared to NT group).

FIGS. 5A-5I: Immunofluorescent staining for de novo regeneration on the quadriceps sagittal section in the injury site of NT group (top row), AT group (middle row) and HA-CS group (bottom row) at 1-week post-surgery. FIG. 5A: Immunofluorescent staining for Pax7 showing very few Pax7 positive cells in the NT group whereas multiple Pax 7 positive cells residing on the newly regenerated muscle fibers were observed surrounding the injury site in FIG. 5B of AT and FIG. 5C of HA-CS groups. FIGS. 5D-5I: Immunofluorescent staining for eMHC illustrating lack of eMHC expression in the (FIGS. 5D, 5G) NT group as compared to enhanced expression of eMHC in the (FIGS. 5E, 5H) AT and (FIGS. 5F, 5I) HA-CS groups at vicinity of injury indicating de novo regeneration of skeletal muscle. White arrows indicate Pax7 positive cells. (Scale bar: 2 mm for FIGS. 5D, 5E, 5F and 200 μm for FIGS. 5A, 5B, 5C, 5G, 5H, 5I).

FIGS. 6A-6L: Masson's trichrome staining in sagittal sections of injured quadriceps for NT group (top row), AT group (middle row) and HA-CS group (bottom row) at 1-week (left two columns) and 4-week (right two columns) post-surgery. (FIGS. 6A, 6B) NT, (FIGS. 6C, 6D) AT, and (FIGS. 6E, 6F) HA-CS groups resulted in minimal to no fibrosis at 1-week post-surgery. Importantly, the HA-CS hydrogel integrated well with the host tissue with no fibrotic tissue capsule observed surrounding the implants. At 4-week post-surgery, dense deposition of collagen was found in (FIGS. 6G, 6H) NT and (FIGS. 6I, 6J) AT groups. Interestingly, minimal collagen deposition was observed at the defect site for (FIGS. 6K, 6L) the HA-CS group. (Scale bar: 2 mm for FIGS. 6A, 6C, 6E, 6G, 6I, 6K and 200 μm for FIGS. 6B, 6D, 6F, 6H, 6J, 6L).

FIGS. 7A-7N: Immunofluorescent staining for CD31 on the sagittal section of injured quadriceps in NT group (top row), AT group (middle row) and HA-CS group (bottom row) at 1-week (left two columns) and 4-week (right two columns) post-surgery. At 1-week post-surgery, CD31 staining at the defect site was sparse for the (FIGS. 7A, 7B) NT group. However, strong CD31 staining was observed in (FIGS. 7C, 7D) the AT group while dense CD31 staining was found surrounding (FIGS. 7E, 7F) the HA-CS implant. A similar trend was observed at 4-week post-surgery for (FIGS. 7G, 7H) NT and (FIGS. 7I, 7J) AT groups. Interestingly, robust CD31 staining was found throughout the defect center for (FIGS. 7K, 7L) the HA-CS implant. White arrows indicate blood vessels. Red—CD31; Blue—Nuclei. (Scale bar: 2 mm for FIGS. 7A, 7C, 7E, 7G, 7I, 7K and 200 μm for FIGS. 7B, 7D, 7F, 7H, 7J, 7L). Immunofluorimetry analysis on the sagittal sections of the injured quadriceps at (FIG. 7M) 1-week and (FIG. 7N) 4-week post-surgery indicating that numbers of CD31 positive blood vessels were significantly higher in HA-CS groups as compared to NT group. (*p<0.05 as compared to NT group).

FIGS. 8A-8N: Immunofluorescent staining for innervation (β-Tubulin III) and AChR (aBTX) in the quadriceps sagittal section for NT (top row), AT (second row) and HA-CS (third row) at 1-week (left two columns) and 4-week (right two columns) post-surgery. (FIGS. 8A, 8B) NT group was associated with very few innervation and AChR positive muscle fibers whereas multiple innervation and AChR positive muscle fibers were found in (FIGS. 8C, 8D) AT and (FIGS. 8E, 8F) HA-CS groups. Interestingly, at 4-week post-surgery innervation and AChR positive muscle fibers were sparse at the injury site for all three groups (FIGS. 8G-8L). This is further corroborated by the quantitation analysis of the AChR clusters for (FIGS. 8M, 8N) 1-week and 4-week post-surgery. There was a significant increase in AChR clusters for HA-CS compared to NT and AT groups at 1-week post-surgery. However, no significant differences were observed among the three groups at 4-week post-surgery. White arrows indicate AChR clusters. (Scale bar: 2 mm for FIGS. 8A, 8C, 8E, 8G, 8I, 8K and 200 μm for FIGS. 8B, 8D, 8F, 8H, 8J, 8L). (* denotes p<0.05 significance from NT group).

The attached drawings are for purposes of illustration and are not necessarily to scale.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In the present disclosure the term “about” can allow for a degree of variability in a value or range, for example, within 10%, within 5%, or within 1% of a stated value or of a stated limit of a range.

In the present disclosure the term “substantially” can allow for a degree of variability in a value or range, for example, within 90%, within 95%, or within 99% of a stated value or of a stated limit of a range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated references should be considered supplementary to that of this document; for irreconcilable inconsistencies, the usage in this document controls.

Parts of this disclosure has been published: Naagarajan Narayanan, et al., “Biomimetic glycosaminoglycan-based scaffolds improve skeletal muscle regeneration in a Murine volumetric muscle loss model”, Bioactive Materials 6 (2021) 1201-1213, the contents of which are hereby incorporated by reference in its entirety.

In some illustrative embodiments, this present disclosure relates to a method for skeletal muscle repair and regeneration for a patient with a volumetric muscle loss comprising the steps of
a. preparing a glycosaminoglycan-based hydrogel;
b. implanting clinically effective amount of hydrogel at the site of muscle loss; and
c. suturing to hold said hydrogel in place.

In some other illustrative embodiments, this present disclosure relates to a method for skeletal muscle repair and regeneration for a patient with a volumetric muscle loss as disclosed herein, wherein said method further comprises a step of incorporating myogenic cells in said hydrogel before the step of implanting.

In some other illustrative embodiments, this present disclosure relates to a method for skeletal muscle repair and regeneration for a patient with a volumetric muscle loss as disclosed herein, wherein said hydrogel for skeletal muscle repair and regeneration comprises hyaluronic acid (HA) with functionalized thiol groups, chondroitin sulfate (CS) with functionalized thiol groups, and poly(ethylene glycol) diacrylate (PEGDA), wherein said HA has a degree of substitution (e.g. functionalization) at about 17% for the thiol groups and said CS has a degree of substitution (e.g. functionalization) at about 47% for the thiol groups, and wherein said HA and said CS are cross-linked by said PEGDA to obtain said hydrogel with a storage modulus G′ of about 13 kPa.

In some other illustrative embodiments, this present disclosure relates to a method for skeletal muscle repair and regeneration for a patient with a volumetric muscle loss as disclosed herein, wherein said hydrogel
a. promotes expression of myogenic markers MyoD, MyoG and MYH8; and
b. supports myoblast proliferation and myogenic differentiation and integration of said hydrogel with its surrounding body tissue of said patient.

In some other illustrative embodiments, this present disclosure relates to a method for skeletal muscle repair and regeneration for a patient with a volumetric muscle loss as disclosed herein, wherein said hydrogel promotes integration with the surrounding host tissue of said patient, facilitates migration of Pax7 positive cells, and promotes de novo myofiber formation, angiogenesis, and innervation.

In some other illustrative embodiments, this present disclosure relates to a method for skeletal muscle repair and regeneration for a patient with a volumetric muscle loss as disclosed herein, wherein said hydrogel minimizes scar tissue formation.

Yet in some other illustrative embodiments, this present disclosure relates to a biomimetic glycosaminoglycan-based hydrogel for skeletal muscle repair and regeneration comprising hyaluronic acid (HA) with functionalized thiol groups, chondroitin sulfate (CS) with functionalized thiol groups, and poly(ethylene glycol) diacrylate (PEGDA), wherein said HA has a degree of substitution (e.g. functionalization) at about 17% for the thiol groups and said CS has a degree of substitution (e.g. functionalization) at about 47% for the thiol groups, and wherein said HA and said CS are cross-linked by said PEGDA to obtain said hydrogel with a storage modulus G′ of about 13 kPa.

In some other illustrative embodiments, this present disclosure relates to a biomimetic glycosaminoglycan-based hydrogel for skeletal muscle repair and regeneration as disclosed herein further comprising cells selected from the group consisting of myogenic cells, nerve cells, stem cells, endothelial cells, adipocytes, fibroblasts, and a combination thereof.

In some other illustrative embodiments, this present disclosure relates to a biomimetic glycosaminoglycan-based hydrogel for skeletal muscle repair and regeneration as disclosed herein, wherein said hydrogel promotes expression of myogenic markers MyoD, MyoG and MYH8, and supports myoblast proliferation and myogenic differentiation and integration of said hydrogel with its surrounding body tissue of said patient.

In some other illustrative embodiments, this present disclosure relates to a biomimetic glycosaminoglycan-based hydrogel for skeletal muscle repair and regeneration as disclosed herein, wherein said hydrogel promotes integration with surrounding host tissue of said patient, facilitates migration of Pax7 positive cells, and promotes de novo myofiber formation, angiogenesis, and innervation.

In some other illustrative embodiments, this present disclosure relates to a biomimetic glycosaminoglycan-based hydrogel for skeletal muscle repair and regeneration as disclosed herein, wherein said hydrogel minimizes scar tissue formation.

In some other illustrative embodiments, this present disclosure relates to a kit for skeletal muscle repair and regeneration comprising a hydrogen comprises hyaluronic acid (HA) with functionalized thiol groups, chondroitin sulfate (CS) with functionalized thiol groups, and poly(ethylene glycol) diacrylate (PEGDA), wherein said HA has a degree of substitution (e.g. functionalization) at about 17% for the thiol groups and said CS has a degree of substitution (e.g. functionalization) at about 47% for the thiol groups, and wherein said HA and said CS are cross-linked by said PEGDA to obtain said hydrogel with a storage modulus G′ of about 13 kPa.

In some other illustrative embodiments, this present disclosure relates to a kit for skeletal muscle repair and regeneration as disclosed herein, wherein said kit further comprising cells selected from the group consisting of myogenic cells, nerve cells, stem cells, endothelial cells, adipocytes, fibroblasts, and a combination thereof.

In some other illustrative embodiments, this present disclosure relates to a kit for skeletal muscle repair and regeneration comprising a hydrogen comprises hyaluronic acid (HA) with functionalized thiol groups, chondroitin sulfate (CS) with functionalized thiol groups, and poly(ethylene glycol) diacrylate (PEGDA), wherein said HA has a degree of substitution (e.g. functionalization) at about 17% for the thiol groups and said CS has a degree of substitution (e.g. functionalization) at about 47% for the thiol groups, and wherein said HA and said CS are cross-linked by said PEGDA to obtain said hydrogel with a storage modulus G′ of about 13 kPa, wherein said hydrogel promotes expression of myogenic markers MyoD, MyoG and MYH8, and supports myoblast proliferation and myogenic differentiation and integration of said hydrogel with its surrounding body tissue of said patient.

In some other illustrative embodiments, this present disclosure relates to a kit for skeletal muscle repair and regeneration comprising a hydrogen comprises hyaluronic acid (HA) with functionalized thiol groups, chondroitin sulfate (CS) with functionalized thiol groups, and poly(ethylene glycol) diacrylate (PEGDA), wherein said HA has a degree of substitution (e.g. functionalization) at about 17% for the thiol groups and said CS has a degree of substitution (e.g. functionalization) at about 47% for the thiol groups, and wherein said HA and said CS are cross-linked by said PEGDA to obtain said hydrogel with a storage modulus G′ of about 13 kPa, wherein said hydrogel promotes integration with surrounding host tissue of said patient, facilitates migration of Pax7 positive cells, and promotes de novo myofiber formation, angiogenesis, and innervation.

In some other illustrative embodiments, this present disclosure relates to a kit for skeletal muscle repair and regeneration comprising a hydrogen comprises hyaluronic acid (HA) with functionalized thiol groups, chondroitin sulfate (CS) with functionalized thiol groups, and poly(ethylene glycol) diacrylate (PEGDA), wherein said HA has a degree of substitution (e.g. functionalization) at about 17% for the thiol groups and said CS has a degree of substitution (e.g. functionalization) at about 47% for the thiol groups, and wherein said HA and said CS are cross-linked by said PEGDA to obtain said hydrogel with a storage modulus G′ of about 13 kPa, wherein said hydrogel minimizes scar tissue formation.

Scaffold-based regenerative engineering offers a promising alternative approach to regenerate damaged or lost tissues [5, 6]. During the tissue regeneration process, the scaffold serves as a temporal extracellular matrix (ECM) that can be gradually replaced by the newly regenerated tissues. An ideal scaffold should be biocompatible, biodegradable, mimic the natural tissue ECM and provide suitable mechanics, present cell-instructive cues to guide cell fate/differentiation and tissue development. The efficacy of scaffold-based regeneration critically depends on the scaffold materials and cell-material interactive properties [7]. Although previous reports with use of decellularized ECM-based scaffolds have showed some promising results, there is limited success to support complete regeneration of VML [8, 9].

Following skeletal muscle injury, the myofibers break down and release their intracellular contents, which triggers a cascade of events that leads to an inflammatory response. Subsequently, cells involved in innervation, blood vessel formation and muscle regeneration migrate to the injury site. To induce de novo skeletal muscle regeneration, Pax7⁺ muscle progenitor cells migrate to the injury site and differentiate to myoblasts that either fuse with existing muscle fibers or with other myoblasts to generate new muscle fibers. Simultaneously, scar tissue is deposited to bridge the gap between the functional muscle fibers to maintain the force transduction along the muscle. However, in severe VML injuries, there is a dense deposition of scar tissue that inhibits the regenerating muscle to bridge the gaps. Therefore, biomaterials that target and orchestrate these natural regenerative processes involving inflammatory response, muscle progenitor cell migration, innervation and blood vessel formation at the injury site could aid in functional regeneration of skeletal muscle.

Hydrogels constitute an attractive scaffold platform for scaffold-based regeneration due to their ECM-mimicking and cell-interactive properties [10, 11]. Natural glycosaminoglycans (GAGs) including hyaluronic acid (HA) and chondroitin sulfate (CS) are important components of skeletal muscle ECM to enable a variety of biological functions. Specifically, hyaluronic acid (HA), a non-sulfated GAG composed of alternating D-glucuronic acid and N-acetyl-D-glucosamine, has been investigated as scaffold materials for skeletal muscle regenerative engineering due to its biocompatibility, biodegradability and its versatility through chemical modifications [9, 12] Similarly, chondroitin sulfate (CS), a sulfated GAG, has been exploited to encourage cellular activities in stem cell niche regulation, angiogenesis, and neural engineering [13-15]. To combine the beneficial properties of these two GAGs, we recently developed a tunable hydrogel system (HA-CS) by combining thiolated HA and thiolated CS cross-linked with poly (ethylene glycol) diacrylate (PEGDA) through thiol-ene click chemistry [16]. The combination of HA and CS offers an ECM-mimicking microenvironment, whereas the selection of PEG is based on its established biocompatibility and chemical versatility in efficient control of hydrogel properties. For example, variations in cross-linking by altering thiol degree of substitution or PEG molecular weight allows for effectively tuning hydrogel physical properties.

The objective of the present study was to develop and characterize a HA-CS hydrogel system with biophysical properties suitable for skeletal muscle regenerative engineering. C2C12 myoblasts were encapsulated within 3D HA-CS hydrogels and characterized for myoblast viability, proliferation, and differentiation. Furthermore, in vivo studies using a murine quadriceps VML model were performed to evaluate the potential of implanted HA-CS hydrogels to facilitate de novo muscle regeneration and restoration of skeletal muscle function. Muscle regeneration was systematically assessed by histology and immunostaining analysis for de novo myofiber formation, fibrosis, angiogenesis, and innervation in combination with treadmill functional analysis.

Results

HA-CS Hydrogel Characterization

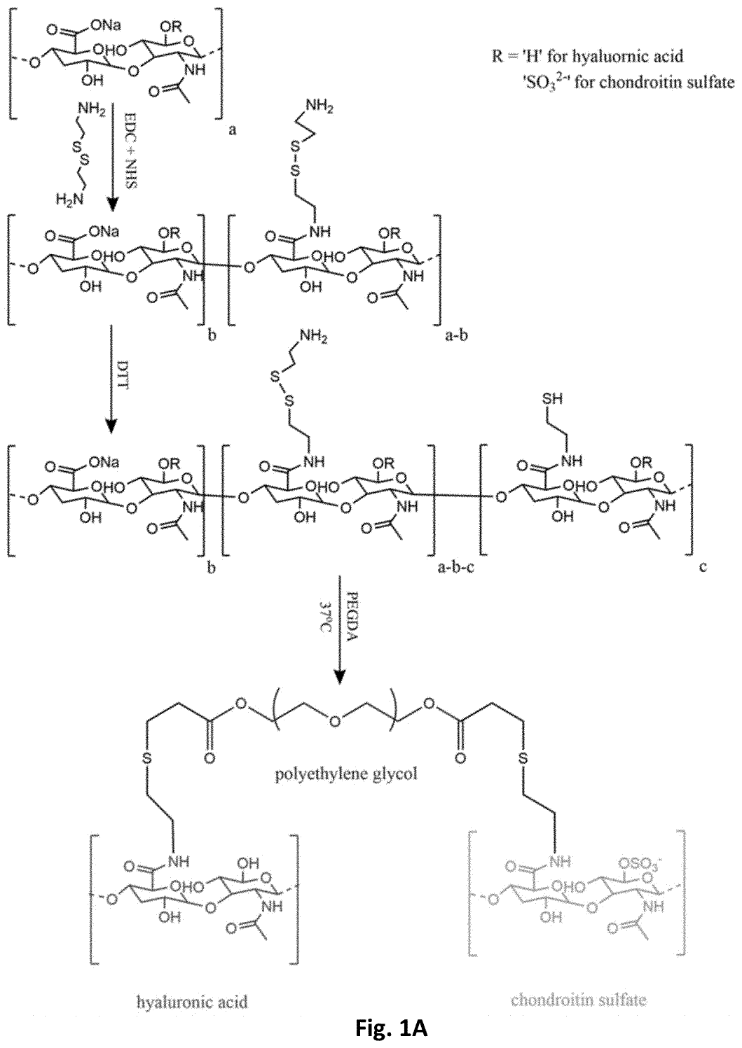

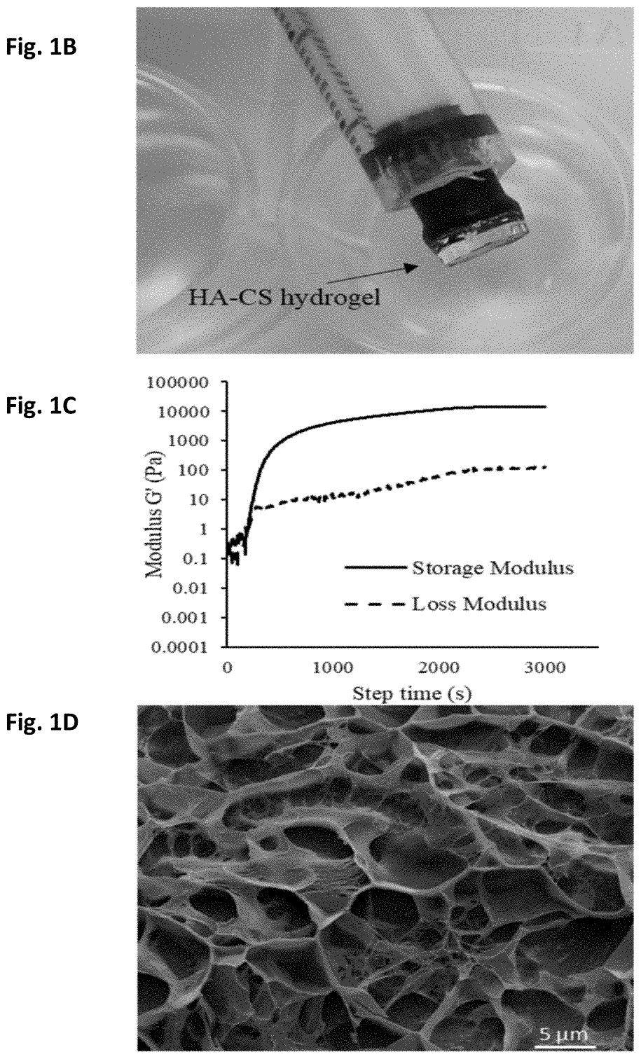

As shown in FIG. 1A, the carboxyl group of HA or CS was first activated by EDC chemistry and further reacted with cystamine dihydrochloride to form the intermediate. The conjugation of cystamine to the backbone of HA or CS was confirmed by the presence of peaks of disulfide pendant signal at 6=2.3-2.8 in H¹ NMR, and the efficiency of the substitution was calculated. In the next step, cystamine present in the backbone of HA or CS was reduced by DTT reaction to introduce thiol groups in the HA or CS backbone as shown in FIG. 1A. The reduction of the disulfide bond of cystamine present in the backbone of HA or CS was confirmed by the decrease in peaks of disulfide pendant signal at 6=2.3-2.8 in H¹ NMR. The reaction efficiency was characterized from H¹ NMR (19% and 49% for HA and CS, respectively), and degree of thiol group substitution (17.5% and 47.2% for HA and CS, respectively) was further confirmed using Elman's assay. Hydrogels were then formed with predetermined concentrations of thiol-modified HA (1% w/v) and CS (7.5% w/v) dissolved in degassed PBS solution with pH of 7.4 and cross-linked with PEGDA at 37° C. The in situ formed hydrogel is shown in FIG. 1B. Rheology characterization of the HA-CS hydrogels with time sweep analysis (FIG. 1C) showed a storage modulus G′ of 13.0±1.8 kPa. The formed HA-CS hydrogels exhibited a porous microstructure as visualized from cryo-SEM micrographs (FIG. 1D).

Myoblast Responses to HA-CS Hydrogels

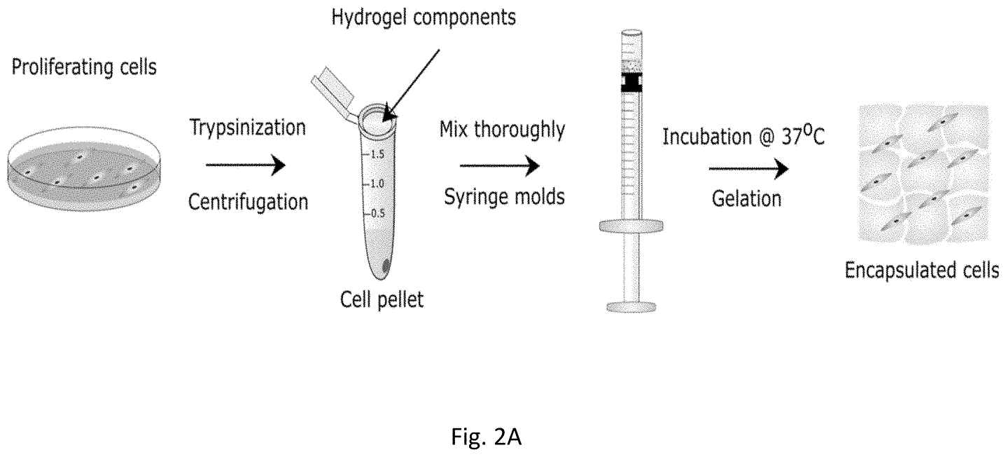

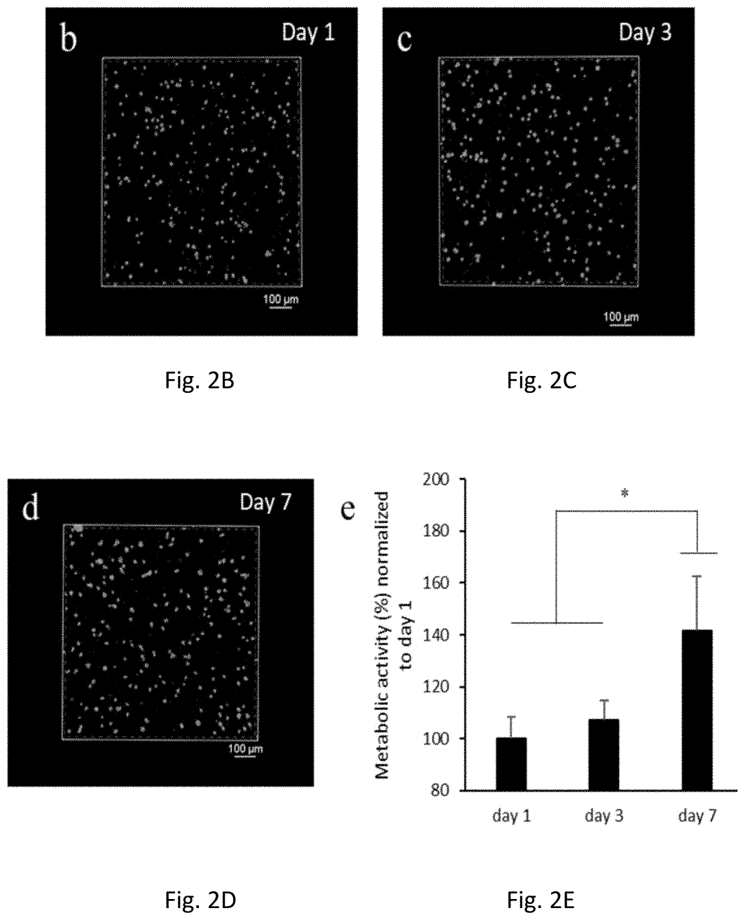

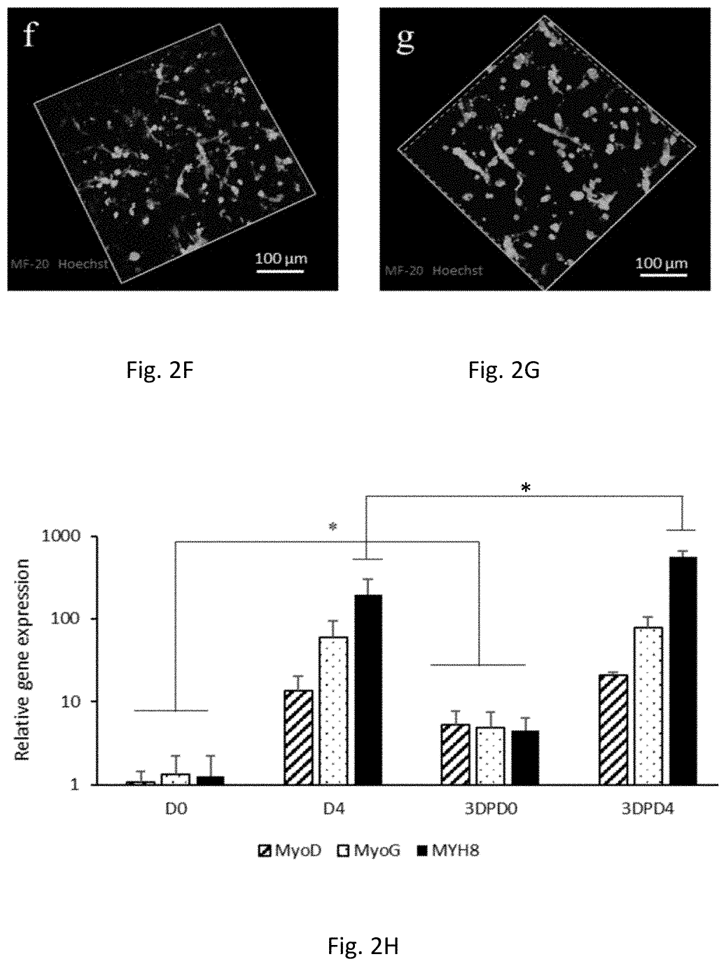

To assess how myoblasts would respond to HA-CS hydrogels, myoblasts were encapsulated in the hydrogel during gelation and characterized for cell viability, cell proliferation, and differentiation (FIG. 2A). Confocal images using a live/dead assay and immunofluorescence staining showed that HA-CS hydrogels supported cell encapsulation and cell culture with high viability (FIG. 2B-2D). MTS assay revealed a significant increase in metabolic activity of encapsulated myoblasts after 7 days of culture, indicating the ability of HA-CS hydrogels to support cell growth as shown in FIG. 2E. Differentiation of the encapsulated myoblasts within hydrogels was analyzed by immunofluorescent staining and qPCR. Positive immunofluorescence staining for MHC indicated that HA-CS hydrogels facilitated myotube formation by the encapsulated myoblasts (FIG. 2F). Interestingly, robust myotube formation was observed when the encapsulated cells were incubated in hydrogels for 3 days in GM as compared to no incubation before switching to DM (FIG. 2G). qPCR results demonstrated a time-dependent increased expression of myogenic differentiation markers of C2C12 cells within the HA-CS hydrogels including MyoD, MyoG and MYH8. Furthermore, incubation of gel-encapsulated C2C12 cells in GM for 3 days (3PD0) resulted in a significant increase in expression of differentiation markers as compared to no incubation (DO) (FIG. 2H). In summary, the HA-CS hydrogels supported the growth of C2C12 cells with high viability and facilitated myoblast to differentiate into myotube; thereby indicating the ability of HA-CS hydrogels to provide a suitable microenvironment for myoblasts.

VML Creation and Hydrogel Implantation

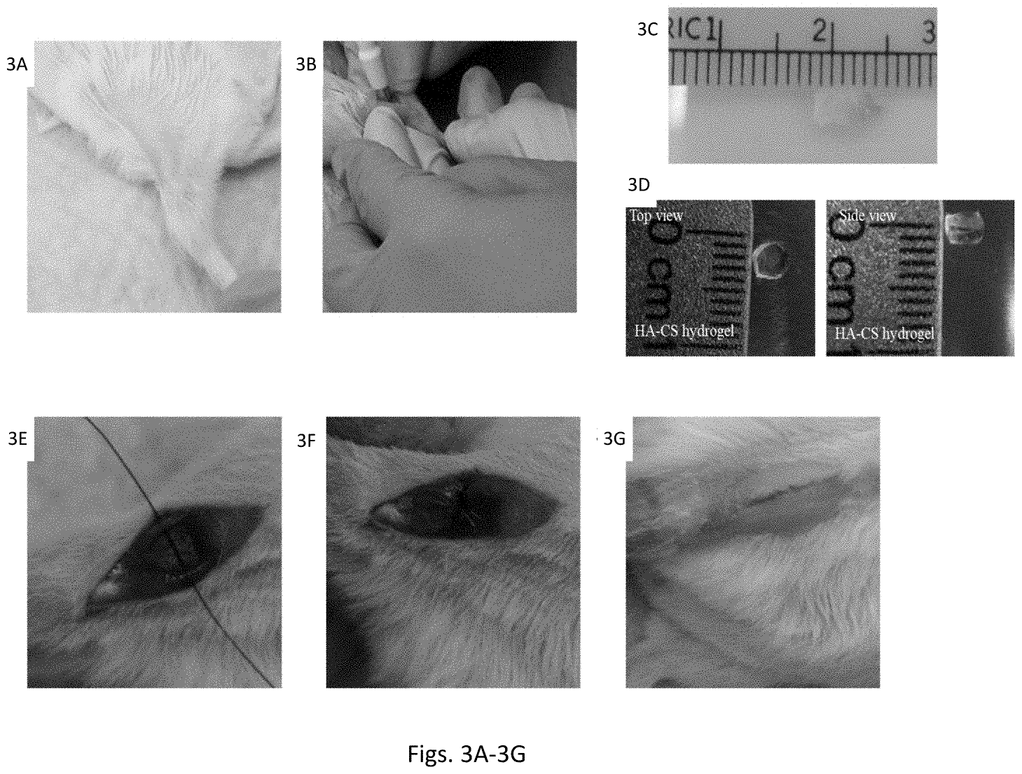

As shown in FIG. 3, VML injury in the quadriceps was surgically created using a 4 mm biopsy punch, which resulted in a full thickness (˜4 mm) defect and ˜17% muscle mass loss. Approximately, 31 mg of muscle mass was surgically removed from each mouse from the quadriceps. The HA-CS hydrogels were then implanted without a cellular payload at the defect site followed by suturing the muscle flap to hold the implants at the defect site (FIG. 3d). None of the animals had post-surgical complications and no mortality was recorded with animals recovering with no macroscopic abnormalities. The excised quadriceps in each treatment group at 4-week post-surgery had comparable weights with respect to their internal controls (76.3±9.9% for NT, 77.1±13.7% for AT, and 76.4±7.3% for HA-CS; n=5), indicating comparable surgical injury creation. Mean body weight for animals at 4-week post-surgery in each group were similar, (29.9±0.6 g for NT, 29.3±1.5 g for AT and 30.7±2.3 g for HA-CS; n=5) suggesting no adverse effects.

Gross Morphology and Histology Analysis of Quadriceps

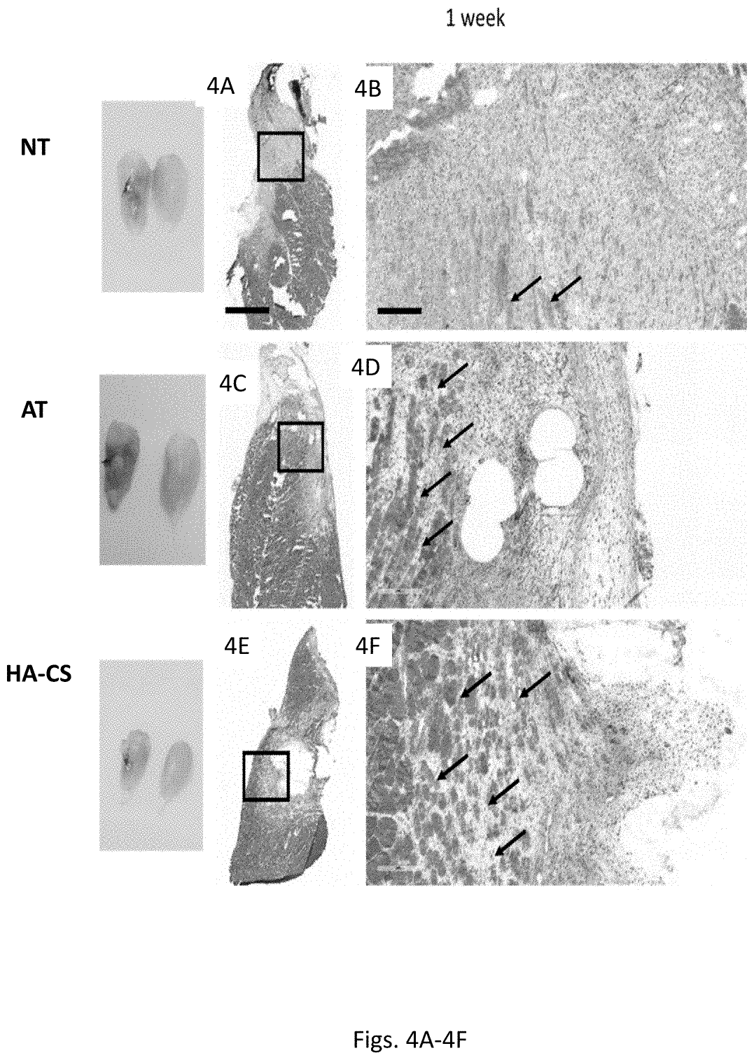

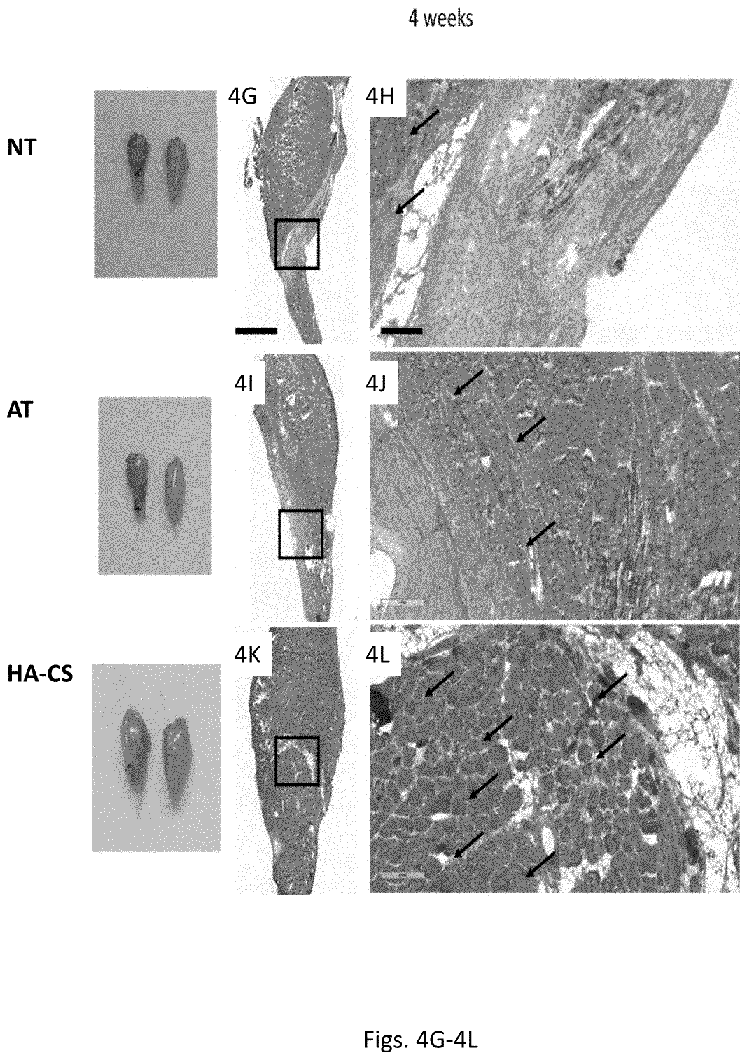

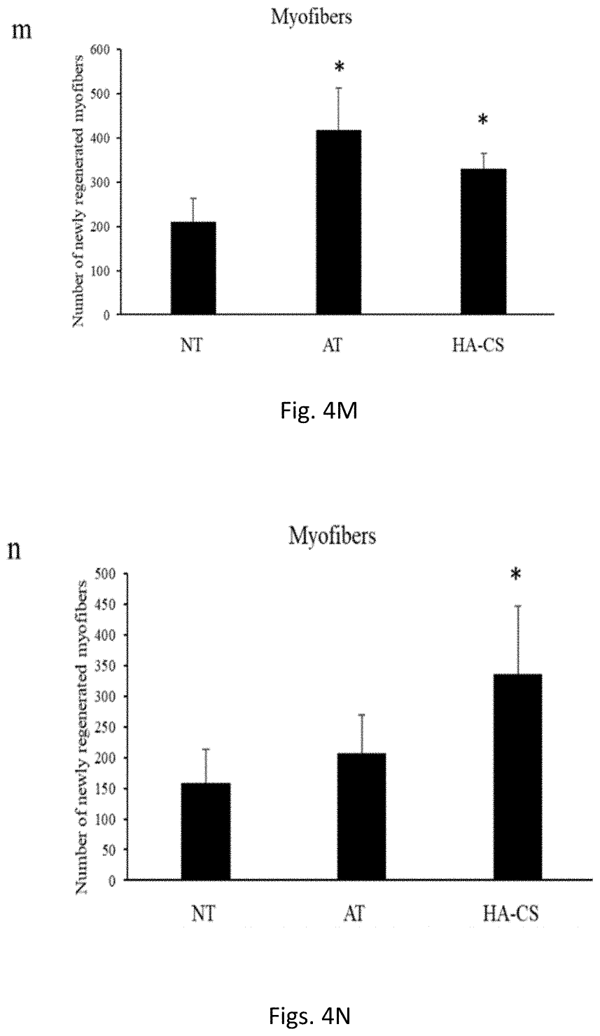

Macroscopically there were no signs of infection, edema or seroma, and the HA-CS implants were well-tolerated with no signs of rejection during 4-week implantation (FIG. 4). Furthermore, there were no macroscopic differences in hydrogel-treated quadriceps compared to internal control (uninjured leg) at 4 weeks post-surgery. Complete degradation of the hydrogel was indicated as no residual polymers were found. In contrast, clear signs of muscle injury were observed for NT and AT groups. H&E staining of the quadriceps sagittal sections revealed formation of newly regenerated muscle fibers with central nuclei in AT and HA-CS groups surrounding the defect site at 1-week post-surgery (FIG. 4a-f) Similar results were obtained when the sagittal sections were immunofluorescently stained for dystrophin, indicating new myofiber formation surrounding the defect site for AT and HA-CS group. Quantification analysis on the full-length sagittal sections revealed a significant increase in number of newly regenerated muscle fibers for AT and HA-CS groups compared to NT group at 1-week post-surgery (FIG. 4m). After 4 weeks of implantation, the defect remained and was clearly identified in the NT group (FIG. 4). The presence of defect at 4-week post-surgery indicated that the endogenous regeneration failed to repair the injury site. However, newly regenerated muscle fibers with central nuclei were found in the defect site for AT and HA-CS groups (FIG. 4g-l). Similar results were observed when the sagittal sections were immunofluorescently stained for dystrophin, which plays a critical role in integrating the cytoskeleton of the muscle cell with the surrounding extracellular matrix. Notably, HA-CS group resulted in a significant increase in number of newly regenerated fibers as compared to NT (FIG. 4n), while no significant differences were observed between AT and NT group at 4-week post-surgery.

De Novo Muscle Regeneration

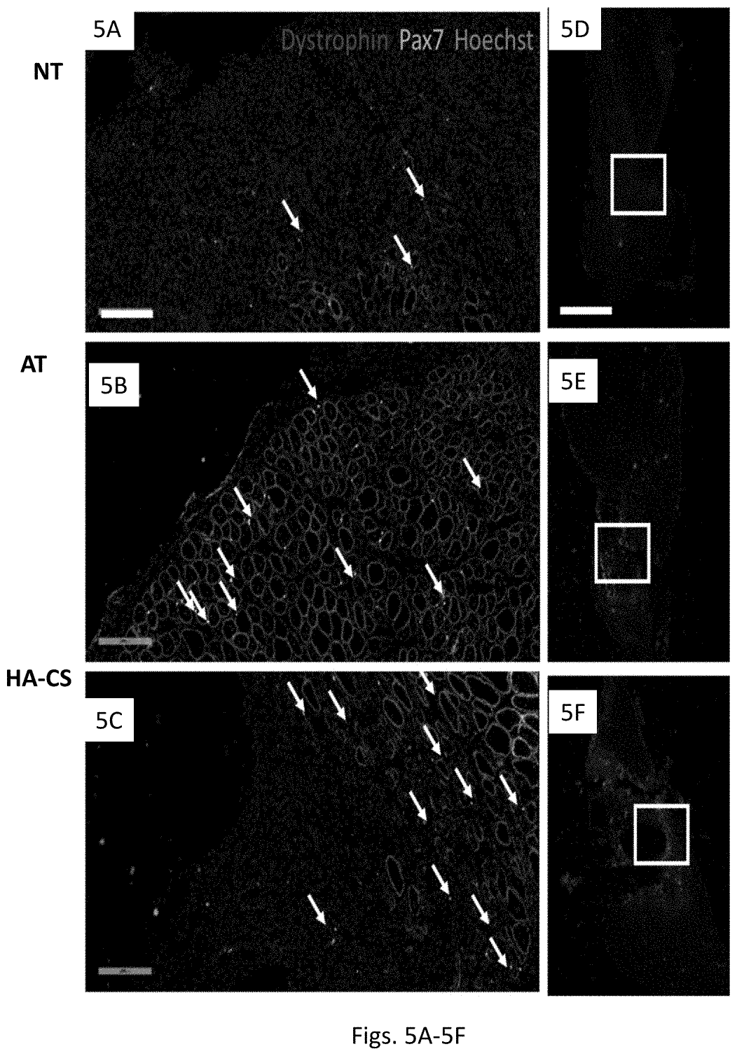

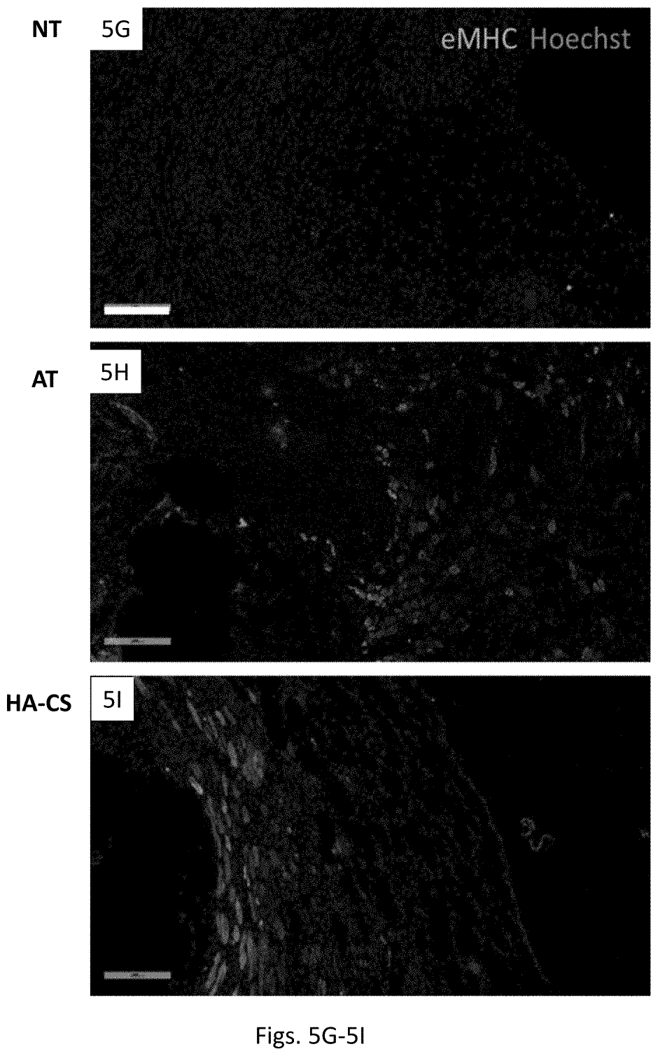

De novo muscle regeneration is critical for successful skeletal muscle repair; thus, we further characterized Pax7 positive cells for satellite cells and eMHC expression for newly formed myofibers in close proximity to the defect site 1-week post-surgery. Negligible population of Pax7 positive cells were noted in NT group whereas numerous Pax7 positive cells were observed surrounding the defect site in AT and HA-CS groups (FIG. 5a-c). Quantification analysis showed a significant increase in Pax7 positive cell numbers for HA-CS group compared to NT group. However, no significant differences were observed between the AT and NT groups. Interestingly, eMHC expression was absent in NT group, whereas eMHC expression was observed surrounding the defect site for AT and HA-CS groups indicating de novo muscle regeneration in proximity to the injury (FIG. 5d-i). This indicated that HA-CS hydrogels promoted migration of Pax7 positive cells, which led to de novo regeneration of skeletal muscle tissue.

Fibrosis in Quadriceps Injury

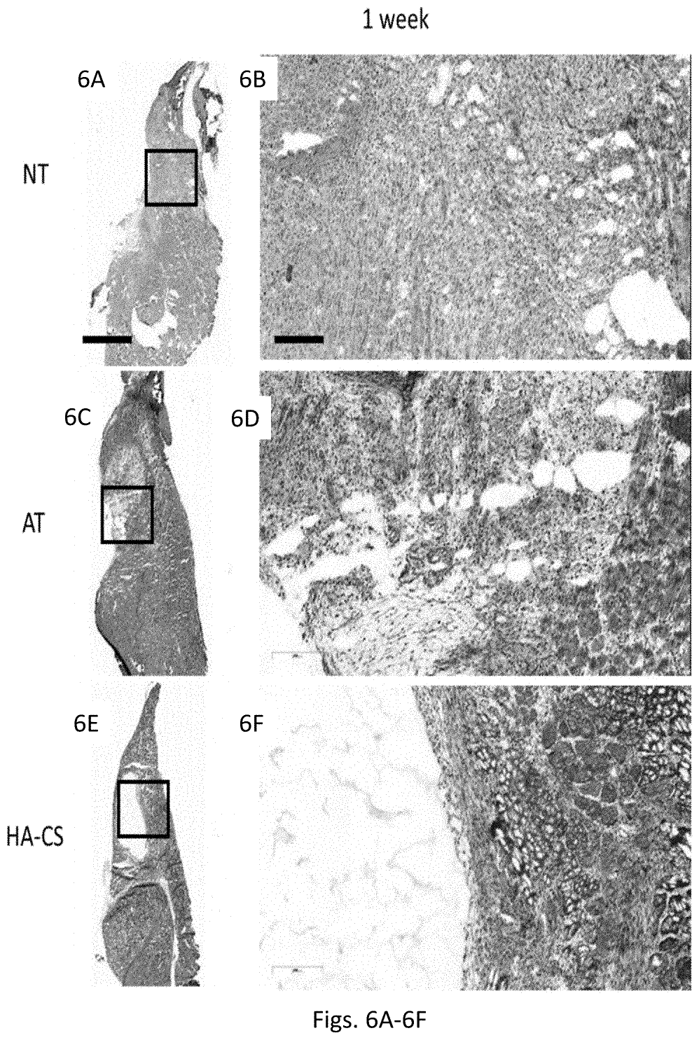

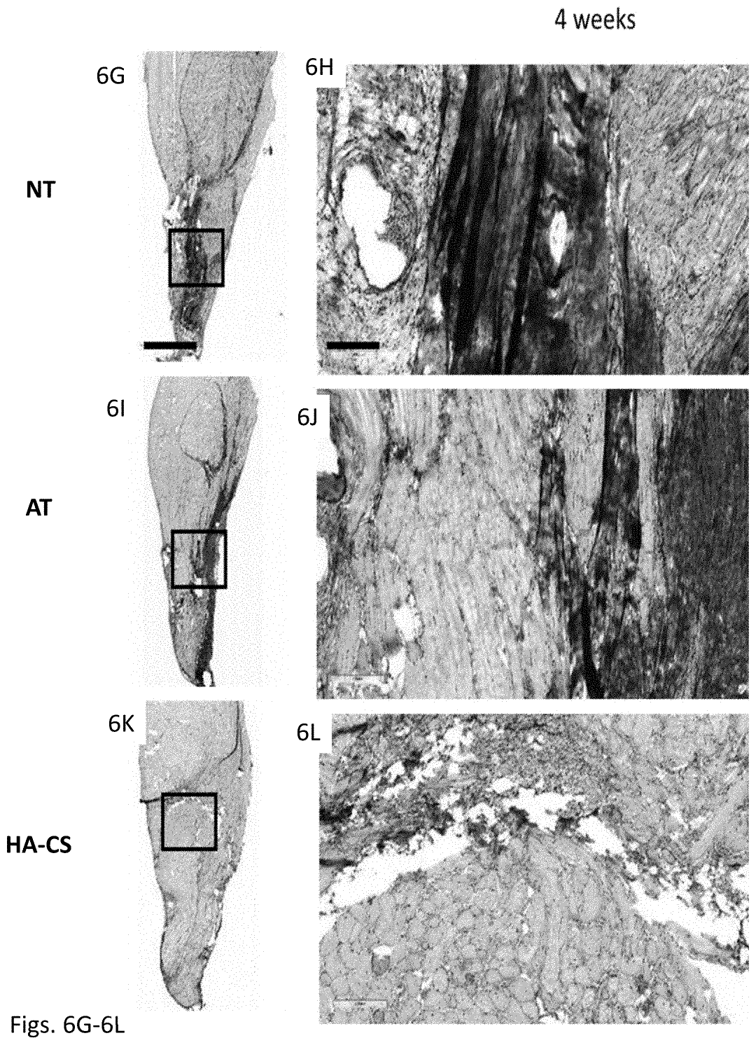

To assess fibrosis associated with hydrogel implantation, we examined the collagen deposition and quantified the fibrosis area at the defect site of injured quadriceps by staining the sagittal sections with Masson's trichrome. At 1-week post-surgery, no evidence for fibrotic tissue formation was observed in the defect site for all three groups (FIG. 6a-f). However, at 4-week post-surgery the injury site was characterized by dense deposition of collagen, leading to fibrotic tissue formation in the NT group (FIG. 6g-h). This further confirmed that the endogenous regeneration system failed to repair the surgically created injury. Collagen deposition was also evident in the AT group at the defect site (FIG. 5i-j). Interestingly, minimal collagen/fibrotic tissue deposition was observed at the defect site for HA-CS group, which indicated that HA-CS hydrogels integrated well with the host tissue supporting muscle regeneration (FIG. 6k-l). Consistently, quantification on the fibrotic tissue area showed a significant decrease in fibrotic tissue for HA-CS group compared to AT and NT groups, indicating HA-CS hydrogels promoted robust muscle regeneration with minimal fibrotic tissue formation.

Vascularization at the Defect Site

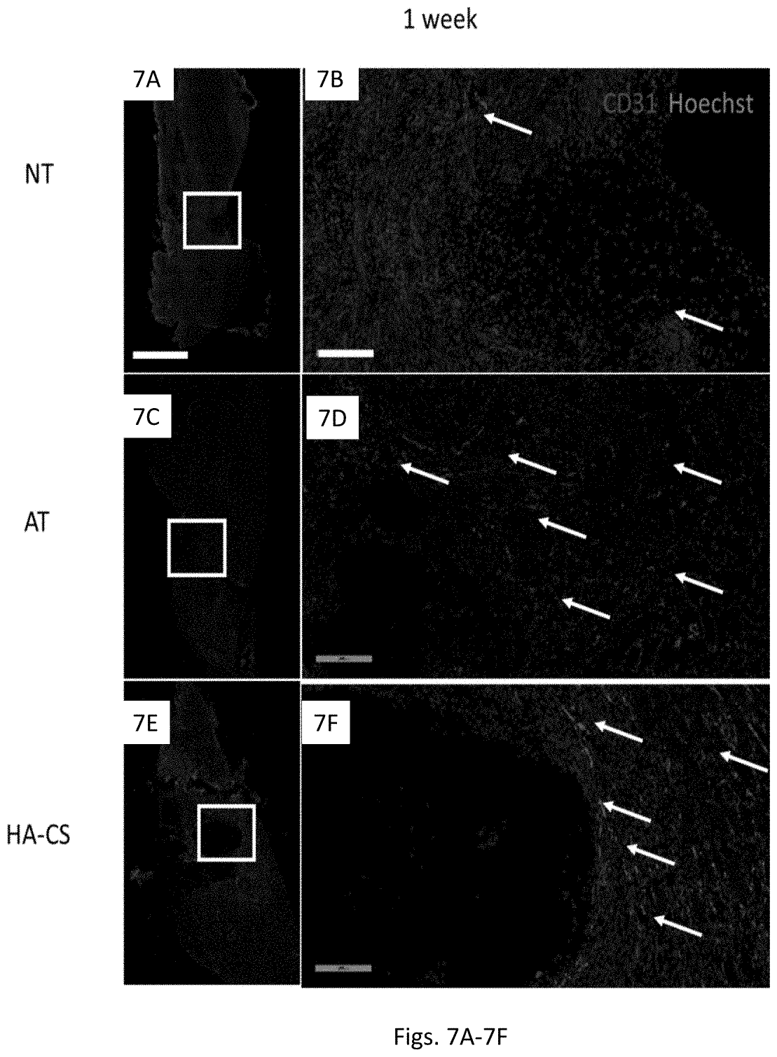

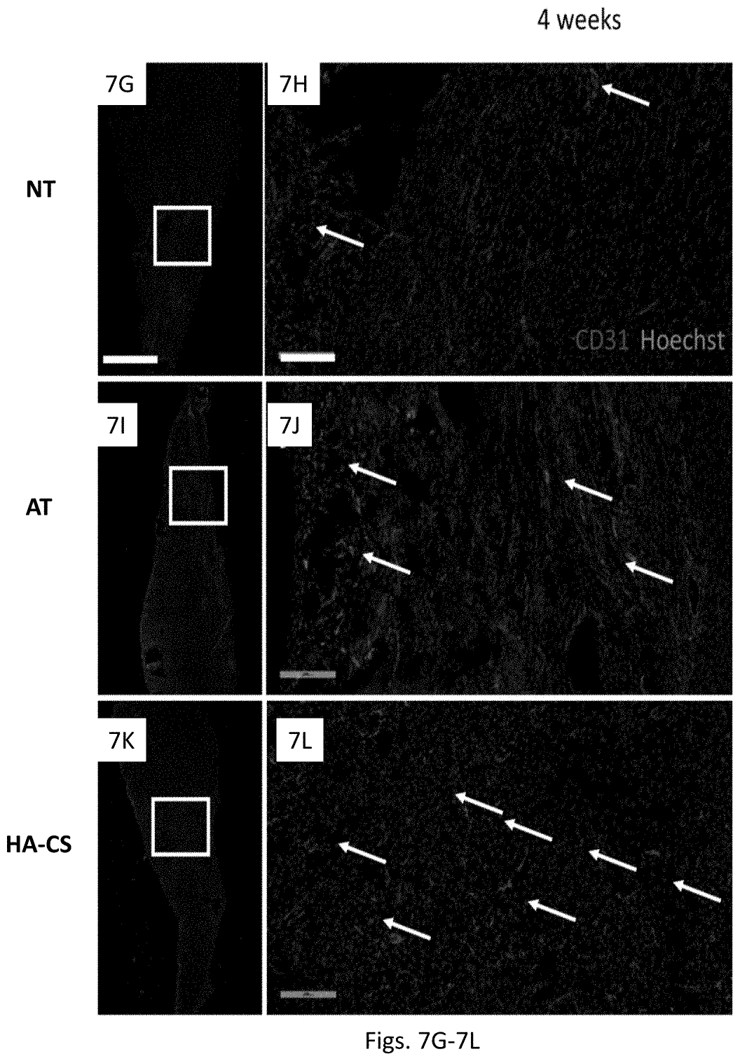

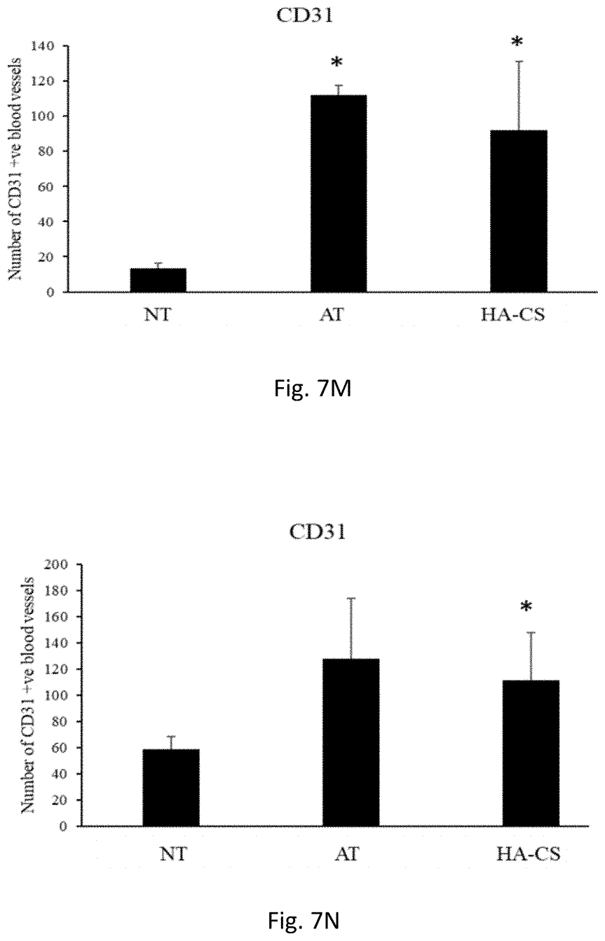

As angiogenesis is an important contributor for regeneration, we assessed the extent of vascularization in the quadriceps defect by immunofluorescently staining the sagittal sections for CD31 positive cells to indicate the presence of endothelial cells. At 1-week post-surgery, scarce presence of CD31 positive cells was observed in NT group (FIG. 7a-b) whereas multiple sites with CD31 positive cells were found in AT and HA-CS groups surrounding the defect, which indicated infiltration of blood vessels (FIG. 6c-f). Quantification analysis on the sagittal sections for CD31 positive cells confirmed a significant increase in blood vessel infiltration for AT and HA-CS group compared to NT group (FIG. 7m). At 4-week post-surgery, CD31 positive cells were absent at the defect site with scarce population surrounding the defect site for NT group (FIG. 6g-h). This further validated lack of sufficient endogenous regeneration to repair the defect. Multiple CD31 positive cell populations were observed for the AT group indicating revascularization surrounding injury with few noticeable areas at the defect site (FIG. 7i-j). Interestingly, numerous CD31 positive cells were observed surrounding the injury and more importantly at the defect site for HA-CS group (FIG. 7k-l). Quantification on sagittal sections demonstrated a significant increase in CD31 positive cell population in and around the injury site for HA-CS group compared to NT group (FIG. 7n), whereas no significant differences were observed between the AT and NT group at 4-week post-surgery. These results were in line with H&E staining suggesting that HA-CS hydrogel facilitated revascularization of the defect during tissue regeneration.

Innervation and Treadmill Functional Analysis

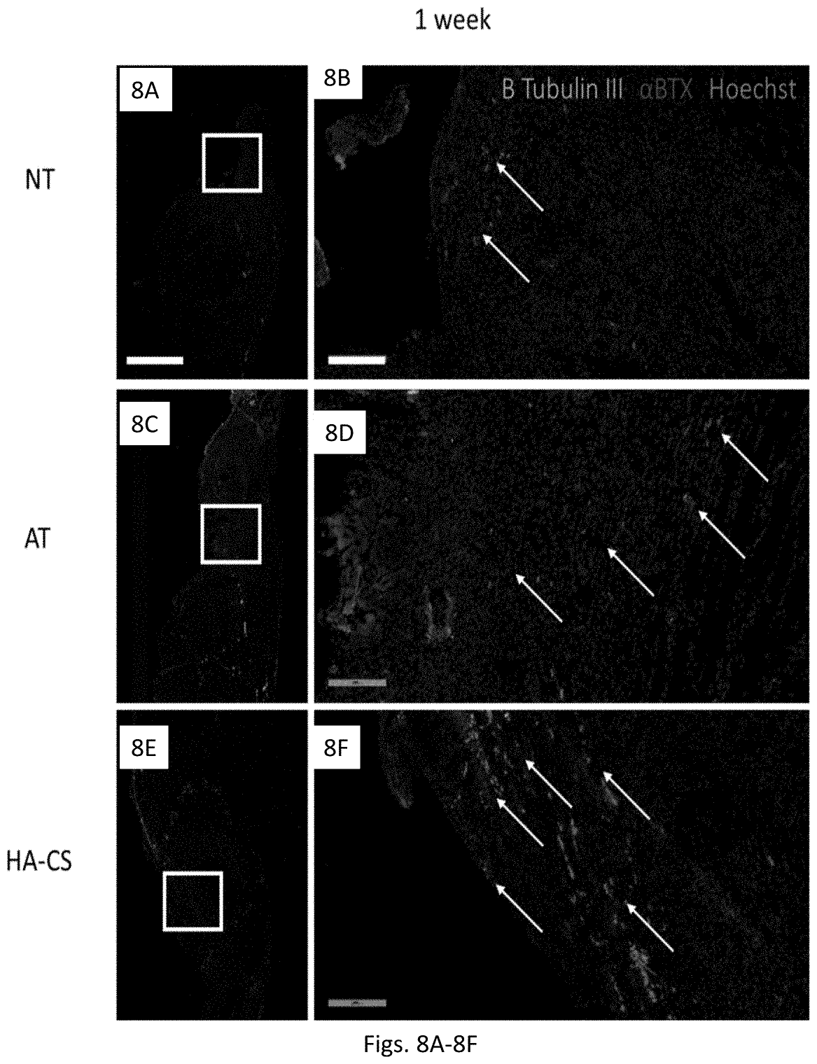

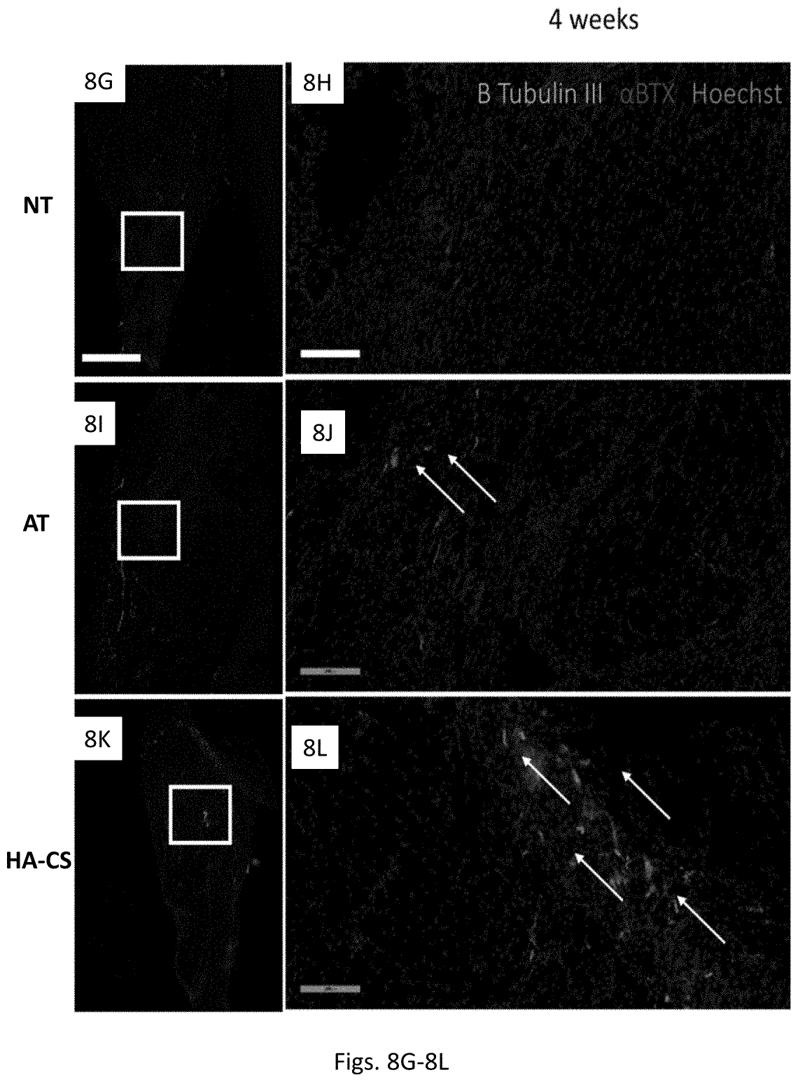

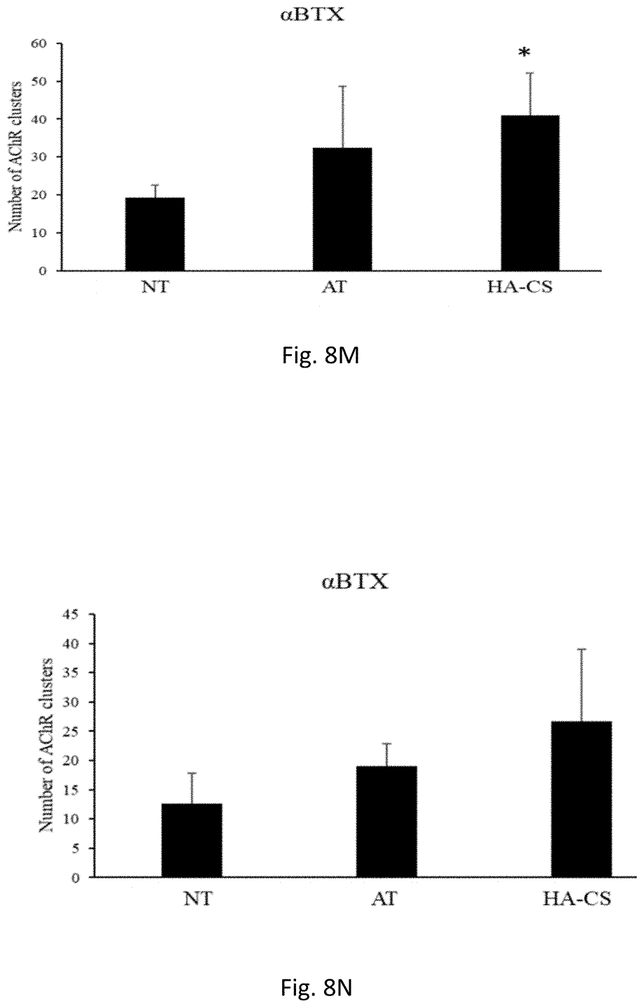

To characterize innervation and neuromuscular junction formation at the injury site, we performed immunofluorescent staining of quadriceps sagittal sections for (3-Tubulin III and AChR to indicate the presence of neurons and neuromuscular junction, respectively. At 1-week post-surgery, multiple innervation and AChR positive myofibers were observed in AT and HA-CS groups in close proximity to the injury site compared to NT group (FIG. 8a-f). Quantification on the number of AChR clusters demonstrated a significant increase in HA-CS group compared to AT and NT groups (FIG. 8m). At 4-week post-surgery, the lack of AChR clustering in the NT group still remained (FIG. 8g-h). In contrast, increased numbers of AChR clusters were observed in close proximity to the injury site for AT (FIG. 8i-j) and HA-CS groups (FIG. 8k-l). A similar trend was observed in quantification analysis on number of AChR clusters among three groups. However, differences were not significant (FIG. 8n). To further muscle performance, we examined recovery of skeletal muscle strength through treadmill fatigue assay, a simple and high throughput method at 1-week and 4-week post-surgery. Running distance for individual animals in each group was recorded and was normalized to their respective baseline values recorded before the surgery. At 1-week post-surgery, recovery rate for animals in AT and HA-CS groups was significantly higher compared to NT group, which indicated early functional improvements. The recovery rate improved with time for all the groups, with the HA-CS group reaching maximum recovery of 85% at 4-week post-surgery. However, no significant differences in recovery rate were observed among the groups. The results were in line with the quantification data on AChR clusters observed at the injury site at 1-week and 4-week post-surgery. These experiments suggested that HA-CS hydrogels, comparable to AT, promoted functional recovery resulting from accelerated tissue regeneration.

### DISCUSSION

Successful scaffold-based tissue regeneration necessitates the design of biomaterials that provide a biocompatible and biomimetic microenvironment to promote cell-material interactions and cellular activities involving cell proliferation and differentiation. Our recent work has demonstrated HA-CS hydrogels are attractive materials for developing cell-instructive scaffolds due to their efficient control of hydrogel properties (e.g., kinetics, gelation time, modulus, and swelling) by simply varying either molecular weight of cross-linker PEGDA or thiol substitution degree. Thiol Michael addition offers unique advantages due to its bioclick nature: high efficiency in aqueous media and lack of by-products as well as the absence of cytotoxic triggers. In this study, we focused on developing HA-CS hydrogels that offer appropriate biophysical cues and cell-material interactions to support myogenic cells and facilitate de novo skeletal muscle regeneration. It has been well documented that mechanical properties of the scaffold play an important role in myoblast behavior [21]. For example, prior studies reported that healthy resting skeletal muscle has an average elastic modulus of 12±4 kPa and optimal myoblast differentiation occurs in tissue-like stiffness [22]. Satellite cells also exhibit enhanced self-renewal and in vivo regeneration at physiological stiffness [23]. Accordingly, we have optimized HA-CS hydrogels via click chemistry using PEGDA3400 to obtain storage modulus G′ of 13.0±1.8 kPa, which is within the range of skeletal muscle tissue. Furthermore, the HA-CS hydrogel scaffold displays a well-defined microporous structure, which has been reported to be beneficial for cell proliferation and ECM deposition [24, 25]. We have shown that the HA-CS hydrogel scaffold supported 3D encapsulation of C2C12 cells with high viability as well as progressive cell proliferation within hydrogels. Additionally, C2C12 cells within the HA-CS hydrogels exhibited an increased expression of myogenic differentiation markers including MyoD, MyoG and MYH8 leading to myotube formation. This is in line with previous report indicating that HA-based hydrogels upregulate myogenic regulatory factors in myoblasts during in vitro culture [9]. Interestingly, we have demonstrated benefits of incubating gel-encapsulated myoblasts in GM for 3 days to achieve enhanced myogenic differentiation as compared to 0-day incubation, which potentially improves myoblast performance through extended myoblast-hydrogel interactions prior to differentiation [26].

VML is a condition where the innate mechanism of the skeletal muscle tissue fails to repair the injury site. VML injuries are characterized by fibrotic tissue deposition, lack of reinnervation, minimal vasculature and insufficient muscle regeneration to bridge the defect site [27]. To understand and characterize biomaterials-based VML regeneration in quadriceps, it is essential to create a surgical defect that mimics VML condition. A recent study reported that a minimum critical size defect of 3-mm full-thickness (with 3-mm biopsy punch and ˜15% muscle loss) was necessary to surgically create a quadriceps VML model in mice [28]. Here, we employed a 4-mm biopsy punch model with ˜17% muscle loss to induce VML in mouse quadriceps. The model employed in this study was in accordance with previous reports validating the use of surgically induced quadriceps VML models, which is characterized by fibrotic tissue deposition, minimal muscle regeneration, and lack of reinnervation and vasculature.

De novo regeneration of skeletal muscle tissue begins with satellite cell activation followed by migration towards the injury site. Subsequently, these cells undergo asymmetric cell division to generate myogenic-committed cells. New muscle fibers are generated as myogenic-committed cells proliferate, differentiate, and fuse with surrounding damaged muscle fibers [27, 29]. Unfortunately, de novo regeneration of skeletal muscles is hindered in severe muscle injuries like VML. Various decellularized ECM-based scaffolds have been explored for treatment of VML injuries due to their off-the-shelf availability. For example, muscle-derived decellularized ECM scaffolds were used for a rat tibialis anterior (TA) muscle VML model but showed limited efficacy in new myofiber generation within the defect site despite a significant increase in isometric force production [30]. It has been also reported in rodent VML studies that there is lack of Pax7 positive cell presence in the VML defect region resulting from insufficient host satellite cell migration when treated with acellular biological scaffolds [31]. In contrast, we have demonstrated the ability of HA-CS hydrogels to encourage satellite cell migration towards the injury site and promote generation of new myofibers expressing eMHC in close proximity to surgically created quadriceps defect at 1-week post-injury. Notably, newly regenerated myofibers were found within the defect site at 4-week post-injury resulting from the HA-CS hydrogel implantation. These encouraging results are consistent with previous reports that have demonstrated the beneficial properties of HA in recruitment of muscle progenitor cells and enhancement of myogenic tissue formation through CD44 and RHAMM signaling [32, 33].

Collagen deposition at the injury site is an important factor to be considered for functional skeletal muscle regeneration. Concurrent deposition of collagen fibers (scar tissue) takes place as new myofibers are being generated. In the case of less severe injuries, the deposited scar tissues act as conduits for facilitating myofiber formation. However, in VML injuries, the fibroblasts infiltrate the injury site and rapidly deposit collagen, which consequently turns into a dense cap of fibrotic tissue that blocks further muscle regeneration [31]. This results in splitting of the muscle groups, which often will not reunite, leaving scar tissues in the middle and resulting in impaired muscle function [27]. Previous research efforts using ECM-based scaffolds for VML repair have shown significant fibrotic tissue deposition post transplantation resulting in limited regeneration efficacy [34]. For a biodegradable polymeric scaffold, the collagen deposition greatly depends on material biocompatiblity, degradation, and physicochemical properties. Here, HA-CS hydrogels promoted new myofiber regeneration at the defect site with minimal collagen deposition and complete scaffold degradation at 4-week post-VML injury. This further testifies biocompatibility and degradation of HA-CS hydrogels, which is beneficial for ECM remodeling and skeletal muscle repair. Furthermore, hydrogel-treated muscle showed a 5-fold and 7-fold decrease in fibrosis area as compared to AT and NT groups, respectively (FIG. 6). Our findings on fibrosis seen in the untreated and autograft-treated muscles are in line with previous investigation using autografts to treat VML in the quadriceps femoris and demonstrate the promise of HA-CS hydrogels for VML treatment [35].

Functional skeletal muscle regeneration also greatly depends on the ability of scaffolds to induce vascularization which helps activate residing satellite cell and facilitate migration of myogenic precursor cells to the site of injury [27]. Prior studies indicate that in vivo myoblast proliferation and differentiation requires nutrient and oxygen sources in proximity of ˜150 um [36]. Vascular reconstruction is also widely used in the clinic to enable vascular perfusion in a muscle flap. To address vascularization, pro-angiogenic cells were used in combination with biomaterials to induce vasculature at the VML defect site [37, 38]. However, VML repair studies with MatriStem™, a porcine urinary bladder matrix (UBM), demonstrated that presence of vascular bed at the injury site did not translate to new myofiber formation [31]. As evidenced from CD31 immunostaining in FIG. 7, HA-CS scaffolds promoted formation of neovascular bed throughout the defect center leading to new myofiber formation due to biological properties of HA and CS involved in angiogenesis [39, 40][17]. Results from our hydrogels also highlight the importance of promoting vascular growth for effective regeneration in the center of the VML defect.

Reestablishing innervations to the newly regenerated muscle fibers is beneficial for restoring function of injured muscles. For example, it has been previously reported that implantation of engineered muscle constructs near the femoral nerve improved the force generation by 5-fold [41]. Formation of mature AChRs at the repair site has been shown to be critical for new myofiber regeneration and functional recovery [42, 43]. McClure et al. demonstrated that presence of mature AChR clusters at the injury site was correlated to the improved functional recovery from VML defects in rat gastrocnemius muscle [44]. In our study, HA-CS hydrogels resulted in a significant increase in innervation and AChR clustering compared to NT group as early as 1 week post-injury. However, no further improvements in innervation were achieved for hydrogel-treated muscles at 4 weeks post-surgery. Nevertheless, efficacy of innervation in HA-CS hydrogel group was in par with clinically relevant autologous muscle transplant group [45]. We further validate muscle performance through a treadmill fatigue assay, which is a simple and high throughput technique to assess muscle regeneration [19]. Maximum distance covered and maximum speed attained constitute prime metrics in analyzing muscle function through the treadmill assay [19]. It should be noted that treadmill assay does not characterize the individual muscle group recovery, and it takes into consideration the overall running performance of the mice, which also include the effects from uninjured forelimbs and hindlimb. Also, treadmill performance may also be due to improvements of non-muscle characteristics such as innervation and blood supply. Here, we have shown that there was a significant increase in functional recovery for HA-CS implanted group compared to NT group at 1-week post-surgery, which is in agreement with our findings on innervation. Albeit the increase in recovery (85% to baseline), HA-CS groups were not significantly different from the NT group at 4-week post-surgery. The increase in recovery over time for the mice in NT group could be attributed to the increase in mechanical stability resulting from the significant fibrotic tissue formation as shown in FIG. 6 [34]. Fibrotic tissue has been previously shown to assist in the force transmission by acting as a bridge between adjacent functional muscle tissue. However, this fibrotic tissue deposition does not lead to complete functional recovery [34]. Whereas, the increase in recovery for the HA-CS group can be attributed to the formation of new muscle tissue with minimal scar tissue as evidenced from the histology analysis. Notably, comparable performance between HA-CS hydrogels and AT groups further attests to the potential of this new implantable hydrogel system as a potential alternative to clinically relevant autografts which are associated with limited availability and donor site morbidity concerns. Nevertheless, potential improvements with HA-CS scaffolds in innervation at the defect site could further enhance skeletal muscle functional recovery. Ongoing work focuses on exploring biological cues that promote neural cell migration and innervation and nerve-muscle interactions which could further improve the regeneration efficacy of HA-CS scaffolds for healing a large muscle defect.

### CONCLUSIONS

In this study, a biomimetic HA-CS hydrogel system was synthesized and characterized for skeletal muscle regeneration applications. The HA-CS hydrogels, composed of thiolated HA and thiolated CS cross-linked with PEGDA, were optimized with appropriate mechanical and structural properties through thiol-ene Michael addition click reactions. In vitro studies with C2C12 cells showed that the HA-CS hydrogels supported 3D cell encapsulation with high viability and myoblast proliferation and differentiation. Furthermore, in vivo evaluation using a murine quadriceps VML model demonstrated that the optimized HA-CS hydrogels integrated well with the surrounding tissue with minimal collagen deposition, facilitated migration of Pax7 positive cells, and supported new myofiber formation during a 4-week implantation. Interestingly, the HA-CS hydrogels enhanced angiogenesis and innervation at the defect and showed similar functional recovery as autologous transplants as evidenced from treadmill tests. Therefore, HA-CS hydrogels are promising biomaterials for development of implantable biomimetic scaffolds for regenerative engineering of skeletal muscle tissues.

Materials and Methods

Polymer Synthesis and Hydrogel Preparation

Thiolation of HA and CS was performed as previously reported [17]. Briefly, sodium salts of HA (2-3 MDa, Carbosynth Limited) (2.5 mmol) or CS (20-30 kDa, Carbosynth Limited) (2 mmol) were dissolved in 2-(N-morpholino) ethanesulfonic acid (MES) buffer (0.1 M MES, 0.1M NaCl, pH 6.0). Then, 1-ethyl-3-(3-dimethylaminopropyl) carbodiimide (EDC) (12.5 mmol) and N-hydroxysuccinimide (NHS) (34 mmol) were added to the reaction flask and allowed to react for 2 h. Then, pH was adjusted to 7.2 with NaOH and cystamine dihydrochloride (20 mmol) was subsequently added and allowed to react overnight. The reaction product was exhaustively dialyzed (MWCO 12-14 kDa) against 0.1M NaCl (60 h), 25% ethanol (12 h) and water (12 h). The dialyzed product was lyophilized and stored at −20° C. till further use. Cystamine conjugated HA or CS was dissolved in degassed water and PBS, respectively, before being reduced with precalculated DL-Dithiotreitol (DTT) at pH 7-8. Thiolated HA or CS was purified by precipitating in ethanol thrice before lyophilizing. The purified product was stored at −80° C. before further use.

Hydrogels were formulated using a previously optimized method [17]. In brief, thiolated HA (1% w/v), CS (7.5% w/v) and PEGDA3400 (Alfa Aesar) (25% w/v) were dissolved separately in sterile, degassed PBS (pH 7.4) under nitrogen condition. Subsequently, individual components with predetermined composition were mixed to obtain the hydrogel solution. The hydrogel solution was gelled by incubating the samples at 37° C.

Rheological Characterization

Rheology experiments were carried out using a Discovery Series Hybrid Rheometer-3 (TA) fitted with a parallel plate (20 mm diameter) in oscillating mode at 37° C. Hydrogel solution was mixed well before loading onto the preheated Peltier stage. Parallel plate geometry was set at 1 mm. A strain rate of 0.1% and frequency of 6.28 rad s⁻¹ was used for time sweep until a plateau of storage modulus G′ was reached.

Cryo-Scanning Electron Microscope (Cryo-SEM) Characterization of Hydrogel

The microporous structure of HA-CS hydrogel was analyzed using a cryo-SEM (FEI Nano Nova SEM; Gatan alto 2500 cryo system). Preformed hydrogels were attached to the SEM stub adaptor and flash frozen in a nitrogen slush (−190° C.). Fracture was introduced using a scalpel in a cryochamber maintained at −130° C. Frozen water at the fractured surface was sublimated at −90° C. before coating the surface with palladium. The fractured surface was imaged at 5 kv at different magnifications.

Cell Culture and Maintenance

C2C12 murine myoblasts were cultured and maintained in growth media (GM) consisting of high glucose DMEM (with L-glutamate and sodium pyruvate) supplemented with 10% fetal bovine serum and 1% penicillin/streptomycin. Routinely, cells were cultured in 10 cm petri plates and cultured in a humidified chamber maintained at 37° C. and 5% CO₂. To differentiate the cells, cells were switched to differentiation media (DM) (consisting of high glucose DMEM supplemented with 2% horse serum and 1% penicillin/streptomycin) once the cells reached required confluence.

Cell Encapsulation and Characterization

Thiolated HA was sterilized with ethanol and stored in sterile conditions before use. Thiolated CS and PEGDA were filtered by passing the individual solutions through 0.22 μm syringe filter. To encapsulate the cells, cells growing in petri dishes were trypsinized and pelleted. Predetermined hydrogel components were added to the cell pellet and mixed thoroughly. For cell viability and proliferation studies, cell density of 5×10⁶ cells per mL of hydrogel solution was used. For differentiation studies, cell density of 5×10⁷ cells per mL of hydrogel was used. 20 μL of hydrogel cell mixture were added to the precut syringe mold and incubated at 37° C. to induce gelation. Encapsulated cells were cultured and maintained in GM (changed once in 2 days) for the cell viability and proliferation studies. Viability of C2C12 cells within hydrogels was characterized with a live/dead cell viability kit (Molecular Probes). In brief, calcein AM and ethidium homodimer were mixed with 1×PBS (LD solution) as per manufacturer instructions. Cell culture media was removed from the cell encapsulated hydrogels and replaced with 500 μL of LD solution to ensure complete coverage. The samples were incubated for 30 min, washed thrice with 1×PBS, and imaged under Nikon-A1R confocal microscope. Cell proliferation was analyzed using 3-(4,5-dimethylthiazol-2-yl)-5-(3-carboxymethoxyphenyl-2-(4-sulfophenyl)-2H-tetrazolium) (MTS, Promega) mitochondrial reduction [18]. In brief, cell culture media was removed from the cell encapsulated hydrogels and washed with 1×PBS thrice before replacing with 4800 μL of MTS assay solution (80 μL of Promega MTS reagent with 400 μL of fresh culture media). The samples were incubated for 2 h before collecting the solution to record the absorbance at 490 nm.

For the differentiation study, encapsulated cells were grown either overnight or for 3 days in GM; they were then switched to DM and maintained for 4 days (changed once in 2 days). Myotube formation was characterized by immunofluorescence staining for the myotube maturation marker, myosin heavy chain (MHC). Briefly, HA-CS hydrogels with encapsulated cells were fixed in 4% paraformaldehyde for 10 mins, followed by quenching for 10 mins in 100 mM glycine. The samples were subsequently incubated in blocking buffer (5% goat serum, 2% bovine serum albumin, 0.1% Triton X-100 and 0.1% sodium azide) for 1 h. Next, samples were incubated in primary antibody (MF-20, Development Studies Hybridoma Bank; 1:20) for overnight at 4° C. Lastly, samples were washed in PBS followed by incubating in secondary antibody and counter staining for nucleus.

Total RNA was extracted using Trizol reagent following the manufacturer's instructions. 2 μg of total RNA was revere transcribed to cDNA using random primers and M-MLV reverse transcriptase (Invitrogen). Quantitative PCR (qPCR) was performed in a Roche Light Cycler 480 PCR system with SYBR green master mix to evaluate gene expression of myogenic markers including MyoD (early-stage marker), MyoG (mid-stage marker), and MYH8 (late-stage marker).

Animal Care and Surgical Procedure

All the procedures involving animals were performed in accordance to Purdue University's animal care and user committee. 2-month-old Male NRG mice were purchased from Purdue Cancer Center facility and used for the study. All the animals were housed in the animal facility and fed with sterile, acidified water (pH 2.7-3) and sterile, standard rodent chow. For the surgical procedure, mice were anesthetized with ketamine-xylazine cocktail. The surgical site (left hind limb, right hind limb kept as internal control) was prepared by removing hair and sterilizing with alcohol pads. A skin incision was made just above the knee joint to expose the quadriceps. The injury to the quadriceps was created by a 4 mm diameter biopsy punch (Sklar Instruments). Suture was initiated at the muscle flap surrounding the injury to identify the injury area. For autograft treatment (AT, n=8) group, the removed muscle was placed back at the defect site, and the initiated suture was used to hold the replaced muscle at the injury site and close the muscle injury. For the hydrogel treatment group (HA-CS, n=8), hydrogels were implanted at the injury site and held together with the initiated suture. For the no treatment group (NT, n=8) the injury was left without any treatment. The skin incision was closed using tissue glue. The wound was covered with betadine and monitored for skin infection.

Specimen Harvest and Gross Morphology of the Quadriceps

At predetermined time point of day 7 (n=3) and day 28 (n=5) post-surgery, animals were sacrificed for further analysis. After euthanasia, skin on the hind limb was gently removed to expose the quadriceps. Proximal and distal end of the injured and non-injured quadriceps were surgically removed and imaged for gross morphology analysis. The isolated quadriceps was flash frozen in optimal cutting temperature compound by submerging in 2-methyl butane cooled with liquid nitrogen. The frozen tissue was further used for histology and immunofluorescence analysis.

Histology and Immunofluorescence Staining

The frozen quadriceps was cryo-sectioned to obtained 5 μm sagittal sections in a Leica CM1860 cryostat. The VML area in the quadriceps was recognized by the presence of sutures that were placed during the surgery; the sutures were also used as an indicator to identify the injury site during cryo-sectioning. For hematoxylin and eosin (H&E) staining, sections were fixed in formalin:ethanol mixture, washed in distilled water and stained with Gill's hematoxylin for 5 minutes. The sections were rinsed in tap water and stained with eosin for 15 seconds. Further, the sections were dehydrated in ethanol and xylene before being sealed in mounting media (MM24, Leica). For Masson's trichrome staining, sections were fixed in Bouin's solution for 1 h and washed with tap water. Further, sections were stained in Weigert's iron hematoxylin solution for 10 minutes, washed in distilled water, stained in Biebrich scarlet-acid fuchsin for 10 minutes, differentiated in phosphomolybdic-phosphotungstic acid solution for 10 minutes, and stained in aniline blue (2.5% in 2% acetic acid) for 20 seconds. Then, samples were washed and dehydrated in ethanol and xylene before being sealed in mounting media (MM24, Leica). For immunofluorescence staining, sections were fixed in 4% paraformaldehyde (PBS) for 15 minutes, quenched in 100 mM glycine for 10 minutes, and blocked in blocking buffer (5% goat serum, 2% bovine serum albumin, 0.1% Triton X-100 and 0.1% sodium azide in PBS) for 1 h. Then, samples were incubated in primary antibody overnight at 4° C. Primary antibodies used: dystrophin (1:1000), pax7 (1:50) (Developmental Studies Hybridoma Bank), MYH3 (1:100) (Developmental Studies Hybridoma Bank), CD31 (1:200) (Abcam), β-Tubulin III (1:1000) (Abcam). Secondary antibodies used: anti-mouse alexa fluor 488 (1:1000) (Life Technologies) and anti-rabbit alexa fluor 555 (1:1000) (Abcam). Sections were stained for acetylcholine receptor (AChR) with α-bungerotoxin conjugated with alexa fluor 647 (Life technologies). Nuclei were counter stained with Hoechst 33342 (1:1000). Histology and immunofluorescence slides were scanned using Leica Aperio system (Versa 8) at 20× magnification. Quantifications for the histology and immunofluorescence slides (n≥3 for each group at each time point) were performed in ImageScope software within the defect region on the sagittal sections of the quadriceps.

Treadmill Functional Analysis

Treadmill functional assay was performed as previously reported [19]. Briefly, mice were trained on a treadmill (Eco 3/6 treadmill; Columbus Instruments, Columbus, Ohio, US) with a fixed angle slope of 10° at a constant speed 10 m/s for 10 minutes for 3 consecutive days. On the day of experiment, mice were allowed to run on a treadmill at a constant speed for 5 minutes, before increasing the speed at a rate of 2 m/min every 2 minutes. The mice were allowed to run until they were exhausted (inability to run for 10 s despite mechanical pushing). Running distance was recorded at tabulated for each individual mouse. Treadmill tests were performed before the surgery for each individual mouse to have a baseline. Further, the treadmill test was performed on day 7 (n=8) and day 28 (n=5) post-surgery. Recovery was calculated by normalizing the running distance of each mouse post-surgery with their respective baseline values. Mice that persisted in refusal to run even after several pushes and electrical stimulation were excluded for a given time point; however, they were reintroduced to treadmill at later time points as mentioned in the previous report [20].

Statistical Analysis

Quantitative data were reported as mean±standard deviation (SD). Statistical analysis was performed using a one-way analysis of variance (one-way ANOVA). Comparison between the two means was determined using the Tukey test with a minimum confidence level of p<0.05 for statistical significance.

Polymer Synthesis and Characterization

Individual steps in the polymer synthesis of thiol free HA and CS were characterized and analyzed in accordance to our research groups previous publication. Degree of substitution was calculated for each step as given below.

Degree of Substitution for Cysteine Conjugation:

Degree of substitution was calculated based on the H1 NMR spectra of the obtained product. Aᵢ denotes the area calculated for the respective peak i.

Degree of Cystamine Substitution:

$$D {S}_{( - C ystamine )} = \frac{\frac{( {A}_{a} + {A}_{b} )}{8}}{( \frac{{A}_{C}}{3} )} \times 100 \% \qquad ( 1 )$$

Degree of Substitution for Thiol Free Groups:

Degree of substitution for thiol free group was characterized and analyzed based on H1 NMR spectra. Further, thiol groups were quantified using Elman's assay. Aᵢ denotes the area calculated for the respective peak i.

Degree of Thiol Free Groups:

$$D {S}_{( thiol free )} = \frac{( \frac{8 \times {A}_{C} \times {DS}_{( - C y s )}}{3} - ( {A}_{a} + {A}_{b} + {A}_{c} + {A}_{d} ) )}{( \frac{{A}_{C}}{3} ) \times 4} \times 100 \% \qquad ( 2 )$$

Degree of thiol free groups were calculated to be 19.5% of HA and 49% for CS. The estimated degree of substitution was validated by Elman's assay (17.5% for HA and 47.2% for CS).

Those skilled in the art will recognize that numerous modifications can be made to the specific implementations described above. The implementations should not be limited to the particular limitations described. Other implementations may be possible.

It is intended that the scope of the present methods and apparatuses be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

### REFERENCES CITED

[1] S. Kuang, K. Kuroda, F. Le Grand, M. A. Rudnicki, Asymmetric self-renewal and commitment of satellite stem cells in muscle, Cell 129 (5) (2007) 999-1010.
[2] A. Mauro, Satellite cell of skeletal muscle fibers, The Journal of biophysical and biochemical cytology 9 (2) (1961) 493.
[3] J. B. Holcomb, L. G. Stansbury, H. R. Champion, C. Wade, R. F. Bellamy, Understanding combat casualty care statistics, Journal of Trauma and Acute Care Surgery 60 (2) (2006) 397-401.
[4] B. T. Corona, J. C. Rivera, J. G. Owens, J. C. Wenke, C. R. Rathbone, Volumetric muscle loss leads to permanent disability following extremity trauma, J Rehabil Res Dev 52 (7) (2015) 785-92.
[5] N. Narayanan, C. Jiang, G. Uzunalli, S. K. Thankappan, C. T. Laurencin, M. Deng, Polymeric electrospinning for musculoskeletal regenerative engineering, Regenerative Engineering and Translational Medicine 2 (2) (2016) 69-84.
[6] N. Narayanan, C. Jiang, C. Wang, G. Uzunalli, N. Whittern, D. Chen, O. G. Jones, S. Kuang, M. Deng, Harnessing Fiber Diameter-Dependent Effects of Myoblasts Toward Biomimetic Scaffold-Based Skeletal Muscle Regeneration, Frontiers in Bioengineering and Biotechnology 8 (2020) 203.
[7] M. Deng, R. James, C. T. Laurencin, S. G. Kumbar, Nanostructured polymeric scaffolds for orthopaedic regenerative engineering, IEEE Trans Nanobioscience 11 (1) (2012) 3-14
[8] C. Fuoco, L. L. Petrilli, S. Cannata, C. Gargioli, Matrix scaffolding for stem cell guidance toward skeletal muscle tissue engineering, Journal of orthopaedic surgery and research 11 (1) (2016) 86.
[9] J. M. Silva Garcia, A. Panitch, S. Calve, Functionalization of hyaluronic acid hydrogels with ECM-derived peptides to control myoblast behavior, Acta Biomater 84 (2019) 169-179.
[10] S. Kumbar, C. Laurencin, M. Deng, Natural and Synthetic Biomedical Polymers, Elsevier, Burlington-San Diego, 2014.
[11] L. S. Nair, C. T. Laurencin, Biodegradable polymers as biomaterials, Progr Polymer Sci 32 (8-9) (2007) 762-798.
[12] S. M. Goldman, B. E. P. Henderson, T. J. Walters, B. T. Corona, Co-delivery of a laminin-111 supplemented hyaluronic acid based hydrogel with minced muscle graft in the treatment of volumetric muscle loss injury, PLoS One 13(1) (2018) e0191245.
[13] A. J. Hayes, D. Tudor, M. A. Nowell, B. Caterson, C. E. Hughes, Chondroitin sulfate sulfation motifs as putative biomarkers for isolation of articular cartilage progenitor cells, Journal of Histochemistry & Cytochemistry 56 (2) (2008) 125-138.
[14] P. Kastana, E. Choleva, E. Poimenidi, N. Karamanos, K. Sugahara, E. Papadimitriou, Insight into the role of chondroitin sulfate E in angiogenesis, The FEBS journal (2019).
[15] A. Conovaloff, A. Panitch, Characterization of a chondroitin sulfate hydrogel for nerve root regeneration, Journal of neural engineering 8 (5) (2011) 056003.
[16] L. Kuang, C. Jiang, G. N. Gellert, N. P. Damayanti, J. Irudayaraj, M. Deng, Bio-inspired composite hydrogels for musculoskeletal regenerative engineering, 10th World Biomaterials Congress doi: 10.3389/conf.FBIOE.2016.01.00193 (2016).
[17] L. Kuang, N. P. Damayanti, C. Jiang, X. Fei, W. Liu, N. Narayanan, J. Irudayaraj, O. Campanella, M. Deng, Bioinspired glycosaminoglycan hydrogels via click chemistry for 3D dynamic cell encapsulation, Journal of Applied Polymer Science 136 (5) (2019) 47212.
[18] M. Deng, S. G. Kumbar, L. S. Nair, A. L. Weikel, H. R. Allcock, C. T. Laurencin, Biomimetic Structures: Biological Implications of Dipeptide—Substituted Polyphosphazene—Polyester Blend Nanofiber Matrices for Load—Bearing Bone Regeneration, Advanced Functional Materials 21 (14) (2011) 2641-2651.
[19] F. Yue, P. Bi, C. Wang, J. Li, X. Liu, S. Kuang, Conditional loss of Pten in myogenic progenitors leads to postnatal skeletal muscle hypertrophy but age-dependent exhaustion of satellite cells, Cell reports 17 (9) (2016) 2340-2353.
[20] M. Svensson, P. Rosvall, A. Boza-Serrano, E. Andersson, J. Lexell, T. Deierborg, Forced treadmill exercise can induce stress and increase neuronal damage in a mouse model of global cerebral ischemia, Neurobiology of stress 5 (2016) 8-18.
[21] B. J. Kwee, D. J. Mooney, Biomaterials for skeletal muscle tissue engineering, Curr Opin Biotechnol 47 (2017) 16-22.
[22] A. J. Engler, M. A. Griffin, S. Sen, C. G. Bonnemann, H. L. Sweeney, D. E. Discher, Myotubes differentiate optimally on substrates with tissue-like stiffness: pathological implications for soft or stiff microenvironments, J Cell Biol 166 (6) (2004) 877-87.
[23] A. Urciuolo, M. Quarta, V. Morbidoni, F. Gattazzo, S. Molon, P. Grumati, F. Montemurro, F. S. Tedesco, B. Blaauw, G. Cossu, G. Vozzi, T. A. Rando, P. Bonaldo, Collagen VI regulates satellite cell self-renewal and muscle regeneration, Nat Commun 4 (2013) 1964.
[24] Z.-K. Cui, S. Kim, J. J. Baljon, B. M. Wu, T. Aghaloo, M. Lee, Microporous methacrylated glycol chitosan-montmorillonite nanocomposite hydrogel for bone tissue engineering, Nature communications 10 (1) (2019) 1-10.
[25] X. Li, Q. Sun, Q. Li, N. Kawazoe, G. Chen, Functional hydrogels with tunable structures and properties for tissue engineering applications, Frontiers in chemistry 6 (2018).
[26] A. S. Salimath, A. J. Garcia, Biofunctional hydrogels for skeletal muscle constructs, J Tissue Eng Regen Med 10 (11) (2016) 967-976.
[27] N. J. Turner, S. F. Badylak, Regeneration of skeletal muscle, Cell Tissue Res 347 (3) (2012) 759-74.
[28] S. E. Anderson, W. M. Han, V. Srinivasa, M. Mohiuddin, M. A. Ruehle, J. Y. Moon, E. Shin, C. L. San Emeterio, M. E. Ogle, E. A. Botchwey, N.J. Willett, Y. C. Jang, Determination of a Critical Size Threshold for Volumetric Muscle Loss in the Mouse Quadriceps, Tissue Eng Part C Methods 25 (2) (2019) 59-70.
[29] R. Ghadiali, S. Guimond, J. Turnbull, A. Pisconti, Dynamic changes in heparan sulfate during muscle differentiation and ageing regulate myoblast cell fate and FGF2 signalling, Matrix Biology 59 (2017) 54-68.
[30] B. T. Corona, X. Wu, C. L. Ward, J. S. McDaniel, C. R. Rathbone, T. J. Walters, The promotion of a functional fibrosis in skeletal muscle with volumetric muscle loss injury following the transplantation of muscle-ECM, Biomaterials 34 (13) (2013) 3324-35.
[31] A. Aurora, J. L. Roe, B. T. Corona, T. J. Walters, An acellular biologic scaffold does not regenerate appreciable de novo muscle tissue in rat models of volumetric muscle loss injury, Biomaterials 67 (2015) 393-407.
[32] S. Calve, J. Isaac, J. P. Gumucio, C. L. Mendias, Hyaluronic acid, HAS1, and HAS2 are significantly upregulated during muscle hypertrophy, American Journal of Physiology-Cell Physiology 303 (5) (2012) C577-0588.
[33] Y. Leng, A. Abdullah, M. K. Wendt, S. Calve, Hyaluronic acid, CD44 and RHAMM regulate myoblast behavior during embryogenesis, Matrix Biology 78 (2019) 236-254.
[34] B. T. Corona, X. Wu, C. L. Ward, J. S. McDaniel, C. R. Rathbone, T. J. Walters, The promotion of a functional fibrosis in skeletal muscle with volumetric muscle loss injury following the transplantation of muscle-ECM, Biomaterials 34 (13) (2013) 3324-3335.
[35] M. T. A. Li, N. J. Willett, B. A. Uhrig, R. E. Guldberg, G. L. Warren, Functional analysis of limb recovery following autograft treatment of volumetric muscle loss in the quadriceps femoris, Journal of biomechanics 47 (9) (2014) 2013-2021.
[36] R. G. Dennis, P. E. Kosnik, Excitability and isometric contractile properties of mammalian skeletal muscle constructs engineered in vitro, In Vitro Cellular & Developmental Biology-Animal 36 (5) (2000) 327-335.
[37] A. Aurora, N. Wrice, T. J. Walters, R. J. Christy, S. Natesan, A PEGylated platelet free plasma hydrogel based composite scaffold enables stable vascularization and targeted cell delivery for volumetric muscle loss, Acta biomaterialia 65 (2018) 150-162.
[38] K. H. Nakayama, M. Quarta, P. Paine, C. Alcazar, I. Karakikes, V. Garcia, O. J. Abilez, N. S. Calvo, C. S. Simmons, T. A. Rando, Treatment of volumetric muscle loss in mice using nanofibrillar scaffolds enhances vascular organization and integration, Communications biology 2 (1) (2019) 170.
[39] J. Lu, F. Guan, F. Cui, X. Sun, L. Zhao, Y. Wang, X. Wang, Enhanced angiogenesis by the hyaluronic acid hydrogels immobilized with a VEGF mimetic peptide in a traumatic brain injury model in rats, Regenerative Biomaterials (2019).
[40] D. Park, Y. Kim, H. Kim, K. Kim, Y.-S. Lee, J. Choe, J.-H. Hahn, H. Lee, J. Jeon, C. Choi, Hyaluronic acid promotes angiogenesis by inducing RHAMM-TGFβ receptor interaction via CD44-PKCδ, Molecules and cells 33 (6) (2012) 563-574.
[41] V. Dhawan, I. F. Lytle, D. E. Dow, Y.-C. Huang, D. L. Brown, Neurotization improves contractile forces of tissue-engineered skeletal muscle, Tissue engineering 13 (11) (2007) 2813-2821.
[42] I. K. Ko, B.-K. Lee, S. J. Lee, K.-E. Andersson, A. Atala, J. J. Yoo, The effect of in vitro formation of acetylcholine receptor (AChR) clusters in engineered muscle fibers on subsequent innervation of constructs in vivo, Biomaterials 34 (13) (2013) 3246-3255.
[43] L. Wang, J. Shansky, H. Vandenburgh, Induced formation and maturation of acetylcholine receptor clusters in a defined 3D bio-artificial muscle, Molecular neurobiology 48 (3) (2013) 397-403.
[44] M. J. McClure, D. J. Cohen, A. N. Ramey, C. B. Bivens, S. Mallu, J. E. Isaacs, E. Imming, Y. C. Huang, M. Sunwoo, Z. Schwartz, B. D. Boyan, Decellularized Muscle Supports New Muscle Fibers and Improves Function Following Volumetric Injury, Tissue Eng Part A 24 (15-16) (2018) 1228-1241.
[45] B. T. Corona, J. C. Rivera, J. C. Wenke, S. M. Greising, Tacrolimus as an adjunct to autologous minced muscle grafts for the repair of a volumetric muscle loss injury, Journal of experimental orthopaedics 4 (1) (2017) 36.

1. A method for skeletal muscle repair and regeneration for a patient with a volumetric muscle loss comprising the steps of
a. preparing a glycosaminoglycan-based hydrogel;
b. implanting clinically effective amount of hydrogel at the site of muscle loss; and
c. suturing to hold said hydrogel in place.

2. The method according to claim 1 further comprising a step of incorporating myogenic cells in said hydrogel before the step of implanting.

3. The method according to claim 1, wherein said hydrogel for skeletal muscle repair and regeneration comprises hyaluronic acid (HA) with functionalized thiol groups, chondroitin sulfate (CS) with functionalized thiol groups, and poly(ethylene glycol) diacrylate (PEGDA), wherein said HA has a degree of substitution (e.g. functionalization) at about 17% for the thiol groups and said CS has a degree of substitution (e.g. functionalization) at about 47% for the thiol groups, and wherein said HA and said CS are cross-linked by said PEGDA to obtain said hydrogel with a storage modulus G′ of about 13 kPa.

4. The method according to claim 1, wherein said hydrogel
a. promotes expression of myogenic markers MyoD, MyoG and MYH8; and
b. supports myoblast proliferation and myogenic differentiation and integration of said hydrogel with its surrounding body tissue of said patient.

5. The method according to claim 1, wherein said hydrogel promotes integration with the surrounding host tissue of said patient, facilitates migration of Pax7 positive cells, and promotes de novo myofiber formation, angiogenesis, and innervation.

6. The method according to claim 1, wherein said hydrogel minimizes scar tissue formation.

7. A biomimetic glycosaminoglycan-based hydrogel for skeletal muscle repair and regeneration comprising hyaluronic acid (HA) with functionalized thiol groups, chondroitin sulfate (CS) with functionalized thiol groups, and poly(ethylene glycol) diacrylate (PEGDA), wherein said HA has a degree of substitution (e.g. functionalization) at about 17% for the thiol groups and said CS has a degree of substitution (e.g. functionalization) at about 47% for the thiol groups, and wherein said HA and said CS are cross-linked by said PEGDA to obtain said hydrogel with a storage modulus G′ of about 13 kPa.

8. The biomimetic glycosaminoglycan-based hydrogel according to claim 7 further comprising cells selected from the group consisting of myogenic cells, nerve cells, stem cells, endothelial cells, adipocytes, fibroblasts, and a combination thereof.

9. The biomimetic glycosaminoglycan-based hydrogel according to claim 7, wherein said hydrogel promotes expression of myogenic markers MyoD, MyoG and MYH8, and supports myoblast proliferation and myogenic differentiation and integration of said hydrogel with its surrounding body tissue of said patient.

10. The biomimetic glycosaminoglycan-based hydrogel according to claim 7, wherein said hydrogel promotes integration with surrounding host tissue of said patient, facilitates migration of Pax7 positive cells, and promotes de novo myofiber formation, angiogenesis, and innervation.

11. The biomimetic glycosaminoglycan-based hydrogel according to claim 7, wherein said hydrogel minimizes scar tissue formation.

12. A kit for skeletal muscle repair and regeneration comprising a hydrogen comprises hyaluronic acid (HA) with functionalized thiol groups, chondroitin sulfate (CS) with functionalized thiol groups, and poly(ethylene glycol) diacrylate (PEGDA), wherein said HA has a degree of substitution (e.g. functionalization) at about 17% for the thiol groups and said CS has a degree of substitution (e.g. functionalization) at about 47% for the thiol groups, and wherein said HA and said CS are cross-linked by said PEGDA to obtain said hydrogel with a storage modulus G′ of about 13 kPa.

13. The kit according to claim 12 further comprising cells selected from the group consisting of myogenic cells, nerve cells, stem cells, endothelial cells, adipocytes, fibroblasts, and a combination thereof.

14. The kit according to claim 12, wherein said hydrogel promotes expression of myogenic markers MyoD, MyoG and MYH8, and supports myoblast proliferation and myogenic differentiation and integration of said hydrogel with its surrounding body tissue of said patient.

15. The kit according to claim 12, wherein said hydrogel promotes integration with surrounding host tissue of said patient, facilitates migration of Pax7 positive cells, and promotes de novo myofiber formation, angiogenesis, and innervation.

16. The kit according to claim 7, wherein said hydrogel minimizes scar tissue formation.
